SEQUENCE LISTING (3 sequences)

SEQ ID NO: 1 - protein, 414 aa, Staphylococcus aureus
MKFSTLSEEEFTNYTKKHFKHYTQSIELYNYRNKINHEAHIVGVKNDKNEVIAACLLTEARIFKFYKYFYSHRGPLLDYFDAKLVCYFFKELSKFIYKNRGVFILVDPYLIENLRDANGRIIKNYNNSVIVKMLGKIGYLHQGYTTGYSNKSQIRWISVLDLKDKDENQLLKEMEYQTRRNIKKTIEIGVKVEDLSIEETNRFYKLFQMAEEKHGFHFMNEDYFKRMQEIYKDKAMLKIACINLNEYQDKLKIQLLKIENEMMTVNRALNENPNSKRNKSKLNQLNMQLSSINNRISKTEELILEDGPVLDLAAALFICTDDEVYYLSSGSNPKYNQYMGAYHLQWHMIKYAKSHNINRYNFYGITGVFSNEADDFGVQQFKKGFNAHVEELIGDFIKPVRPILYKFAKLIYKV

SEQ ID NO: 2 - protein, 419 aa, Staphylococcus aureus
MKFTELTVTEFDNFVQNPSLESHYFQVKENIVTRENDGFEVVLLGIKDDNNKVIAASLFSKIPTMGSYVYYSNRGPVMDFSDLGLVDYYLKELDKYLQQHQCLYVKLDPYWLYHLYDKDIVPFEGREKNDALVNLFKSHGYEHHGFTTEYDTSSQVRWMGVLNLEGKTPETLKKTFDSQRKRNINKAINYGVKVRFLERDEFNLFLDLYRETEERAGFVSKTDDYFYNFIDTYGDKVLVPLAYIDLDEYVLKLQQELNDKENRRDQMMAKENKSDKQMKKIAELDKQIDHDQHELLNASELSKTDGPILNLASGVYFANAYEVNYFSGGSSEKYNQFMGPYMMHWFMINYCFDNGYDRYNFYGLSGDFTENSEDYGVYRFKRGFNVQIEELIGDFYKPIHKVKYWLFTTLDKLRKKLKK

SEQ ID NO: 3 - protein, 421 aa, Staphylococcus aureus
MEKMHITNQEHDAFVKSHPNGDLLQLTKWAETKKLTGWYARRIAVGRDGEVQGVAQLLFKKVPKLPYTLCYISRGFVVDYSNKEALNALLDSAKEIAKAEKAYAIKIDPDVEVDKGTDALQNLKALGFKHKGFKEGLSKDYIQPRMTMITPIDKNDDELLNSFERRNRSKVRLALKRGTTVERSDREGLKTFAELMKITGERDGFLTRDISYFENIYDALHEDGDAELFLVKLDPKENIAKVNQELNELHAEIAKWQQKMETSEKQAKKAQNMINDAQNKIAKNEDLKRDLEALEKEHPEGIYLSGALLMFAGSKSYYLYGASSNEFRDFLPNHHMQYTMMKYAREHGATTYDFGGTDNDPDKDSEHYGLWAFKKVWGTYLSEKIGEFDYILNQPLYQLIEQVKPRLTKAKIKISRKLKRK

(19) United States
(12) Patent Application Publication
(10) Pub. No.: US 2002/0072105 A1
(43) Pub. Date: Jun. 13, 2002

(54) Crystallization and structure determination of FemA and FemA-like proteins

(76) Inventors: Timothy E. Benson, Kalamazoo, MI (US); Donald Bryan Prince, Parchment, MI (US)

Correspondence Address: MUETING, RAASCH & GEBHARDT, P.A., P.O. BOX 581415, MINNEAPOLIS, MN, 55458, US

(21) Appl. No.: 09/932,474

(22) Filed: Aug. 17, 2001

Related U.S. Application Data
(60) Provisional application No. 60/226,239, filed on Aug. 17, 2000.
(60) Provisional application No. 60/226,269, filed on Aug. 17, 2000.

(51) Int. Cl. G06F 19/00; G01N 33/48; G01N 33/50; C12N 9/50

(52) U.S. Cl. 435/219; 702/19

(57) ABSTRACT

The x-ray crystal structure of FemA and/or FemA-like proteins is useful for solving the structure of other molecules or molecular complexes, and identifying and/or designing modifiers of FemA activity.

This application claims the benefit of U.S. Provisional Application Ser. Nos. 60/226,239 and 60/226,269, both filed Aug. 17, 2000, both of which are incorporated herein by reference in their entireties.

This application incorporates by reference the material contained on the duplicate (2) compact discs submitted herewith. Each disc contains the following files:

| Name | Size | Contents | Date of File Creation |
|---|---|---|---|
| table_1.txt | 270 KB | Table 1 | August 10, 2001 |

### FIELD OF THE INVENTION

This invention relates to the crystallization and structure determination of Staphylococcus aureus FemA (S. aureus FemA).

### BACKGROUND OF THE INVENTION

Over the past decade, the problem of antibiotic resistant bacterial infections has increased the need for novel antibacterial agents. In particular, methicillin resistant Staphylococcus aureus (S. aureus) has surfaced as one focus of the development of new antibiotics. The resistance of S. aureus to methicillin and other β-lactams comes from the acquisition by the bacteria of another penicillin binding protein PBP2′ (or PBP2a). Through a series of genetic efforts, other genes have been identified as factors involved in methicillin resistance. These factors known as fem (factors essential for methicillin resistance) or aux (auxiliary) genes are involved in cell wall biosynthesis or synthesis of factors associated with the cell wall. Three of these genes, FemX (or FmhB), FemA, and FemB play a vital role in the later stages of peptidoglycan biosynthesis in Staphyloccoci.

Peptidoglycan biosynthesis begins with the formation of UDP-N-acetylmuramic acid from UDP-N-acetylglucosamine by the enzymes MurA and MurB. Formation of lactyl ether stem of UDP-N-acetylmuramic acid creates the locus upon which a five residue peptide chain is built. Construction of this pentapeptide is catalyzed in a nonribosomal fashion by the enzymes MurC, MurD, MurE, and MurF in both Gram negative and Gram positive bacteria. The resulting UDP-N-acetylmuramyl pentapeptide is subsequently attached to an undecaprenyl lipid moiety by MraY and joined to another sugar, UDP-N-acetylglucosamine by MurG. In Staphylococci the next steps of peptidoglycan biosynthesis involve another family of enzymes, FemX, FemA, and FemB which create a pentaglycine strand in a stepwise fashion on the amino terminus of the lysine side chain. FemX is the factor responsible for addition of the first glycine to the -amino group of the lysine side chain, FemA adds the next two glycine residues, and FemB adds the final two glycine residues (FIG. 1). This extended Lys-Gly₅ chain serves as the interstrand bridge between nearby peptide strands. It is thought that the glycines utilized by the Fem proteins come from glycyl-tRNA^{Gly}. Crosslinking between strands can then occur between the lysine-pentapeptide bridge and the carbonyl of the fourth residue (D-Ala) with release of the terminal D-Ala in a transpeptidation step catalyzed by penicillin binding proteins.

Since crosslinking between the peptide strands is critical for stability of the bacterial cell wall to changes in osmotic pressure, FemX, FemA, and FemB are potential targets for the development of antibacterial agents. Genetic analysis of these genes has shown that FemX and FemA are essential for viability of S. aureus, while FemB might not be essential. FemB being non-essential is consistent with a glycyl-peptide bridge of some minimal length (e.g., 2-3 residues) allowing sufficient interstrand crosslinking to maintain cell viability. In addition, the sequences of the three fem genes are closely related and probably share the same general protein fold (FIG. 2).

A need exists in the art for potential small molecule modifiers, and methods for identifying small molecule modifiers, that could modify the activity of more than one member of the FemX, FemA, and FemB family.

### SUMMARY OF THE INVENTION

In one aspect the present invention provides a crystal of S. aureus FemA, preferably having the orthorhombic space group symmetry P2₁2₁2₁. Preferably, the crystal includes a unit cell having dimensions a, b, and c; wherein a is about 40 Å to about 70 Å, b is about 75 Å to about 105 Å, and c is about 95 Å to about 125 Å; and α=β=γ=90°. Preferably, the crystal includes atoms arranged in a spatial relationship represented by the structure coordinates listed in Table 1. Preferably, the crystal includes amino acids having the sequence SEQ ID NO: 1, with one or more methionine optionally being replaced with selenomethionine.

In another aspect, the present invention provides a method for crystallizing an S. aureus FemA molecule or molecular complex. The method includes preparing purified S. aureus FemA at a concentration of about 1 mg/ml to about 50 mg/ml and crystallizing S. aureus FemA from a solution including about 1 wt. % to about 50 wt. % PEG, 0 wt. % to about 50 wt. % glycerol, 0 M to about 1 M NaCl, 0 wt. % to about 40 wt. % DMSO, about 100 mM to about 1 M Ca(OAc)₂ and/or MgCl₂, and buffered to a pH of about 7 to about 10.

In another aspect, the present invention provides a molecule or molecular complex including at least a portion of an S. aureus FemA or S. aureus FemA-like binding site or substrate binding surface. The binding site or substrate binding surface includes amino acids listed in Tables 2, 3, or 4. The binding site or substrate binding surface is defined by a set of points having a root mean square deviation of less than about 1.5 Å from points representing the backbone atoms of the amino acids as represented by the structure coordinates listed in Table 1. Preferably, the binding site is a binding site for coenzyme A.

In another aspect, the present invention provides a molecule or molecular complex that is structurally homologous to an S. aureus FemA molecule or molecular complex. The S. aureus FemA molecule or molecular complex is represented by at least a portion of the structure coordinates listed in Table 1.

In another aspect, the present invention provides a scalable three dimensional configuration of points. In one embodiment, at least a portion of the points, and preferably all of the points, are derived from structure coordinates listed in Table 1 of at least a portion of an S. aureus FemA molecule or molecular complex that includes at least one of a FemA or FemA-like binding site or substrate binding surface. Preferably, at least a portion of the points derived from the S. aureus FemA structure coordinates are derived from structure coordinates representing the locations of at least the backbone atoms of amino acids defining an S. aureus FemA binding site or substrate binding surface, with the binding site or substrate binding surface including the amino acids listed in Tables 2, 3, or 4. Optionally, the points may be displayed as a holographic image, a stereodiagram, a model or a computer-displayed image.

In another embodiment, the scalable three dimensional configuration of points includes at least a portion of the points derived from structure coordinates of at least a portion of a molecule or a molecular complex that is structurally homologous to an S. aureus FemA molecule or molecular complex and includes at least one of an S. aureus FemA or S. aureus FemA-like binding site or substrate binding surface.

In another aspect, the present invention provides a machine-readable data storage medium including a data storage material encoded with machine readable data. When using a machine programmed with instructions for using the data, the machine displays a graphical three-dimensional representation of a molecule or molecular complex. The molecule or molecular complex may be a molecule or molecular complex including at least a portion of an S. aureus FemA or S. aureus FemA-like binding site or substrate binding surface including the amino acids listed in Tables 2, 3, or 4. The substrate binding surface is defined by a set of points having a root mean square deviation of less than about 1.5 Å from points representing the backbone atoms of the amino acids as represented by structure coordinates listed in Table 1. Alternatively, the molecule or molecular complex may be a molecule or molecular complex that is structurally homologous to an S. aureus FemA molecule or molecular complex, with the S. aureus FemA molecule or molecular complex being represented by at least a portion of the structure coordinates listed in Table 1.

In another aspect, the present invention provides a machine-readable data storage medium including a data storage material encoded with a first set of machine readable data. When combined with a second set of machine readable data and using a machine programmed with instructions for using the first set of data and the second set of data, the machine determines at least a portion of the structure coordinates corresponding to the second set of machine readable data. The first set of data includes a Fourier transform of at least a portion of the structure coordinates for S. aureus FemA listed in Table 1 and the second set of data includes an x-ray diffraction pattern of a molecule or molecular complex of unknown structure.

In another aspect, the present invention provides a method for obtaining structural information about a molecule or a molecular complex of unknown structure. The method includes crystallizing the molecule or molecular complex; generating an x-ray diffraction pattern from the crystallized molecule or molecular complex; and applying at least a portion of the structure coordinates set forth in Table 1 to the x-ray diffraction pattern to generate a three-dimensional electron density map of at least a portion of the molecule or molecular complex whose structure is unknown.

In another aspect, the present invention provides a method for homology modeling an S. aureus FemA homolog. The method includes aligning the amino acid sequence of an S. aureus FemA homolog with an amino acid sequence of S. aureus FemA (SEQ ID NO: 1) and incorporating the sequence of the S. aureus FemA homolog into a model of S. aureus FemA formed from structure coordinates set forth in Table 1 to yield a preliminary model of the S. aureus FemA homolog;

subjecting the preliminary model to energy minimization to yield an energy minimized model; and remodeling regions of the energy minimized model where stereochemistry restraints are violated to yield a final model of the S. aureus FemA homolog.

In another aspect, the present invention provides a computer-assisted method for identifying a potential modifier of S. aureus FemA activity. The method includes supplying a computer modeling application with a set of structure coordinates of a molecule or molecular complex, the molecule or molecular complex including at least a portion of an S. aureus FemA or S. aureus FemA-like binding site or substrate binding surface, the substrate binding surface including the amino acids listed in Tables 2, 3, or 4; supplying the computer modeling application with a set of structure coordinates of a chemical entity; and determining whether the chemical entity is expected to bind to or interfere with the molecule or molecular complex. Binding to or interfering with the molecule or molecular complex is indicative of potential modification of S. aureus FemA activity. Preferably, the binding site or substrate binding surface includes the amino acids listed in Tables 2, 3, or 4, with the binding site or substrate binding surface being defined by a set of points having a root mean square deviation of less than about 1.5 Å from points representing the backbone atoms of the amino acids as represented by structure coordinates listed in Table 1. Preferably, determining whether the chemical entity is expected to bind to or interfere with the molecule or molecular complex includes performing a fitting operation between the chemical entity and a binding site or substrate binding surface of the molecule or molecular complex, followed by computationally analyzing the results of the fitting operation to quantify the association between, or the interference with, the chemical entity and the binding site. Optionally, the method further includes screening a library of chemical entities. Optionally, the method further includes supplying or synthesizing the chemical entity, then assaying the chemical entity to determine whether it modifies S. aureus FemA activity.

In another aspect, the present invention provides a computer-assisted method for designing a potential modifier of S. aureus FemA activity. The method includes supplying a computer modeling application with a set of structure coordinates of a molecule or molecular complex, the molecule or molecular complex including at least a portion of an S. aureus FemA or S. aureus FemA-like binding site or substrate binding surface, the binding site or substrate binding surface including the amino acids listed in Tables 2, 3, or 4; supplying the computer modeling application with a set of structure coordinates for a chemical entity; evaluating the potential binding or interfering interactions between the chemical entity and the binding site or substrate binding surface of the molecule or molecular complex; structurally modifying the chemical entity to yield a set of structure coordinates for a modified chemical entity; and determining whether the modified chemical entity is expected to bind to or interfere with the molecule or molecular complex. Preferably, the binding site or substrate binding surface includes the amino acids listed in Tables 2, 3, or 4, with the substrate binding surface being defined by a set of points having a root mean square deviation of less than about 1.5 Å from points representing the backbone atoms of the amino acids as represented by structure coordinates listed in Table 1.

In another aspect, the present invention provides a computer-assisted method for designing a potential modifier of S. aureus FemA activity de novo. The method includes supplying a computer modeling application with a set of structure coordinates of a molecule or molecular complex, the molecule or molecular complex including at least a portion of an S. aureus FemA binding site or substrate binding surface, wherein the binding site or substrate binding surface includes the amino acids listed in Tables 2, 3, or 4; computationally building a chemical entity represented by set of structure coordinates; and determining whether the chemical entity is expected to bind to or interfere with the molecule or molecular complex. Preferably, the binding site or substrate binding surface includes the amino acids listed in Tables 2, 3, or 4, with the binding site or substrate binding surface being defined by a set of points having a root mean square deviation of less than about 1.5 Å from points representing the backbone atoms of the amino acids as represented by structure coordinates listed in Table 1.

In another aspect, the present invention provides a method for making a potential modifier of S. aureus FemA activity including chemically or enzymatically synthesizing a chemical entity to yield a potential modifier of S. aureus FemA activity. In one embodiment, the chemical entity is identified during a computer-assisted process including supplying a computer modeling application with a set of structure coordinates of a molecule or molecular complex, the molecule or molecular complex including at least a portion of a S. aureus FemA or S. aureus FemA-like binding site or substrate binding surface; supplying the computer modeling application with a set of structure coordinates of a chemical entity; and determining whether the chemical entity is expected to bind to or interfere with the molecule or molecular complex at a binding site or substrate binding surface. In another embodiment, the chemical entity is designed during a computer-assisted process including supplying a computer modeling application with a set of structure coordinates of a molecule or molecular complex, the molecule or molecular complex including at least a portion of a S. aureus FemA or S. aureus FemA-like binding site or substrate binding surface; supplying the computer modeling application with a set of structure coordinates for a chemical entity; evaluating the potential binding interactions between the chemical entity and a binding site or substrate binding surface of the molecule or molecular complex; structurally modifying the chemical entity to yield a set of structure coordinates for a modified chemical entity; and determining whether the chemical entity is expected to bind to or interfere with the molecule or molecular complex at the binding site. In still another embodiment, the chemical entity is designed during a computer-assisted process including supplying a computer modeling application with a set of structure coordinates of a molecule or molecular complex, the molecule or molecular complex including at least a portion of a S. aureus FemA or S. aureus FemA-like binding site or substrate binding surface; computationally building a chemical entity represented by set of structure coordinates; and determining whether the chemical entity is expected to bind to or interfere with the molecule or molecular complex at a binding site or substrate binding surface, wherein binding to or interfering with the molecule or molecular complex is indicative of potential modification of S. aureus FemA activity.

In another aspect, the invention provides a potential modifier of S. aureus FemA activity, a composition including a potential modifier of S. aureus FemA activity, or a pharmaceutical composition including a potential modifier of S. aureus FemA activity. The potential modifier or composition is identified, designed, or made according to one or more of the above described methods.

Table 1 lists the atomic structure coordinates for molecule S. aureus FemA as derived by x-ray diffraction from a crystal of that complex. Column 2 lists a number for the atom in the structure. Column 3 lists the element whose coordinates are measured. The first letter in the column defines the element. Column 4 lists the type of amino acid. Column 5 lists a number for the amino acid in the structure. Columns 6-8 list the crystallographic coordinates X, Y, and Z respectively. The crystallographic coordinates define the atomic position of the element measured. Column 9 lists an occupancy factor that refers to the fraction of the molecules in which each atom occupies the position specified by the coordinates. A value of “1 ” indicates that each atom has the same conformation, i.e., the same position, in all molecules of the crystal. Column 10 lists a thermal factor “B” that measures movement of the atom around its atomic center.

TABLE 2
Residues found along the L-shaped channel - the substrate binding
site.

| | | | |
|---|---|---|---|
| His 22 | Glu 141 | Phe 318 | Asn 362 |
| Phe 23 | Thr 143 | Phe 319 | Phe 363 |
| Thr 24 | Phe 149 | Val 326 | Tyr 364 |
| His 29 | Asp 150 | Tyr 327 | Gly 365 |
| Leu 32 | Val 152 | Tyr 328 | Glu 375 |
| Lys 33 | Leu 153 | Ala 329 | Asp 376 |
| Tyr 38 | Gln 154 | Gly 330 | Val 379 |
| Thr 59 | Ile 155 | Gly 331 | Phe 382 |
| Val 61 | Arg 205 | Thr 332 | Lys 383 |
| Val 63 | Met 208 | Arg 337 | Tyr 386 |
| Tyr 69 | Phe 224 | Ser 342 | Ile 398 |
| Tyr 71 | Tyr 225 | Tyr 343 | Tyr 406 |
| Asn 73 | Arg 228 | Val 345 | Tyr 409 |
| Tyr 104 | Tyr 232 | Gln 346 | |
| His 106 | Phe 317 | Tyr 361 | |

TABLE 3
Residues near the coenzyme A binding site in subdomain 1A.

| | | | |
|---|---|---|---|
| Met 18 | Ile 78 | Gln 83 | Asn 128 |
| Pro 19 | Asn 79 | Leu 111 | Trp 130 |
| Tyr 20 | Tyr 80 | Pro 112 | Phe 131 |
| Val 77 | Glu 81 | Gly 127 | |

TABLE 4
Residues near the coenzyme A binding site in subdomain 1B.

| | | | |
|---|---|---|---|
| Leu 178 | Gly 331 | Ser 342 | Phe 382 |
| Arg 179 | Thr 332 | Tyr 343 | Lys 383 |
| Asn 182 | Ser 333 | Phe 363 | Lys 384 |
| Lys 185 | Asn 334 | Val 379 | |
| Tyr 327 | Arg 337 | Lys 381 | |

Abbreviations

The following abbreviations are used throughout this disclosure:

Staphylococcus aureus (S. aureus)

Dimethyl sulfoxide (DMSO)

Dithiothreitol (DTT)

Isopropylthio-β-D-galactoside (IPTG)

Polyethylene glycol (PEG)

Multiple anomalous dispersion (MAD)

Root mean square (r.m.s.)

Root mean square deviation (r.m.s.d.)

The following abbreviations are used for amino acids throughout this disclosure:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = lie = Isoleucine | N = Asn = Asparagine |
| P = Pro = Proline | Q = Gin = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

### BRIEF DESCRIPTION OF THE FIGURES

FIG. 1 is a schematic representation of the biochemical pathway for synthesis of the pentaglycine chain attached to the amino terminus of the lysine side chain by FemX (FmhB), FemA, and FemB.

FIG. 2 illustrates the alignment of protein sequences for FemX (FmhB, SEQ ID NO:3), FemA (SEQ ID NO:1), and FemB (SEQ ID NO:2). Underlined blocks indicate identical residues in all three sequences, underlined bolded blocks indicate identical residues in two of the three sequences, and shaded blocks indicate similar residues. This alignment was conducted in Vector NTI (InforMax, Inc. North Bethesda, Md.).

FIG. 3 depicts a section of the electron density maps for residues 103 to 109 of S. aureus FemA. a) Electron density from solvent flattened multiple anomalous dispersion phases. b) Final 2Fo-Fc electron density.

FIG. 4 illustrates an overview of the three-dimensional structure of S. aureus FemA. The N-terminus is starred and the location of the C-terminus is indicated by an arrow.

FIG. 5 is a schematic depiction of a secondary structure diagram for S. aureus FemA.

FIG. 6 depicts the localization of the peptdioglcyan substrate binding channel. The two arrows mark entry points into an L-shaped channel on the surface of S. aureus FemA.

FIG. 7(a) depicts a stereoview of a superposition of the helical arms of Thermus thermophilus seryl-tRNA-synthetase (Cusack et al., EMBO J., 15:2834-42 (1996)) and S. aureus FemA.

FIG. 7(b) depicts a stereoview of a superposition of the same structures with seryl-tRNA present.

FIG. 7(c) depicts a stereoview of a superposition of seryl-tRNA with S. aureus FemA.

FIG. 8 illustrates a stereo view of a superposition of S. aureus FemA (thin black lines) with Tetrahymena GCN5 (thick gray lines) bound to coenzyme A (location indicated by an arrow) and a histone H3 peptide (omitted for clarity) (pdb id 1qsn). a) Superposition on subdomain 1 from FemA. The r.m.s deviation for the superposition was 1.53A for residues 1-6, 8-14, 40-47, 51-62, 69-73, 84-108, and 397-401 from S. aureus FemA and residues A49-A54, A63-A69. A94-A101, A105-A116, A120-A124, A136-A160, and A198-A202 from Tetrahymena GCN5. b) Superposition on subdomain 2 from FemA. The r.m.s. deviation for the superposition was 1.67 Å for residues 191-197, 200-208, 236-243, 311-321, 324-337, 339-362, 378-386, 157-163 from S. aureus FemA and residues A49-A55, A65-A73, A94-A101, A105-A115, A120-A133, A164-A172, A196-A202 A202 from Tetrahymena GCN5.

FIG. 9 illustrates a stereo view of a superposition of common secondary structure elements in the two subdomains of the globular domain of S. aureus FemA. Residues from subdomain 1 (1-17, 28-34, 40-47, 52-62, 69-78, 85-107, 398-401) from one molecule of S. aureus FemA (black) were superimposed on residues from subdomain 2 (191-208, 223-229, 236-243, 312-322, 324-333, 341-363, 160-167) from another molecule of S. aureus FemA (gray) with an r.m.s. deviation of 2.4 Å.

FIG. 10 depicts the localization of the disaccharide hexapeptide binding channel based on the peptide binding site for GCN5. a) Superposition of GCN5 with the H3 peptide (location indicated by an arrow) and FemA. b,c) Surface representations of FemA showing the localization of the H3 peptide (the view in c is rotated 90° around the horizontal axis).

FIG. 11 is a schematic representation of the “Coenzyme A Shuttle” model for S. aureus FemA function (see text for details). In this diagram the first glycine attached by FemX (FmhB) is already shown on the peptidoglycan (circled). The two new glycines added by FemA are starred.

FIG. 12 is a schematic representation of the “tRNA transfer” model for S. aureus FemA function (see text for details). In this diagram the first glycine attached by FemX (FmhB) is already shown on the peptidoglycan (circled). The two new glycines added by FemA are starred.

### DETAILED DESCRIPTION OF THE PREFERRED EMBODIMENTS

Crystalline Form(s) and Method of Making

Applicants have produced crystals including S. aureus FemA (and substrate or inhibitor), which are suitable for x-ray crystallographic analysis. The three-dimensional structure of S. aureus FemA or S. aureus FemA/ligand complex was solved using high resolution x-ray crystallography. Preferably, a crystal has orthorhombic space group P2₁2₁2₁. The crystallized enzyme is a monomer and has one molecule in the asymmetric unit. Preferably, the crystal includes rectangular shaped unit cells, each unit cell having the dimensions a=55±15 Å, b=90±15 Å, and c=110±15 Å. More preferably, a crystal includes rectangular shaped unit cells, each unit cell having the dimensions a=55±5 Å, b=90±5 Å, and c=110±5 Å. Most preferably, S. aureus FemA is crystallized in orthorhombic space group P2₁2₁2₁ with cell constants a=53.9 Å, b=90.4 Å, c=109.3 Å, and α=β=γ=90° C.

Accordingly, one embodiment of the invention provides a method for preparing an S. aureus FemA or S. aureus FemA/ligand crystal. The method includes preparing purified S. aureus FemA at a concentration of about 1 mg/mil to about 50 mg/ml; and crystallizing S. aureus FemA from a solution including about 1 wt. % to about 50 wt. % PEG, 0 wt. % to about 50 wt. % glycerol, 0 M to about 1 M NaCl, 0 wt. % to about 40 wt. % DMSO, about 100 mM to about 1 M Ca(OAc)₂ and/or MgCl₂, and buffered to a pH of about 7 to about 10. Preferably, crystals of selenomethionine S. aureus Fem A are grown in 10% PEG 8000, 0.1 M imidazole, pH 8.0, 0.2 M Ca(OAc)₂. Preferably, the crystals are frozen in liquid nitrogen for data collection. Variation in buffer and buffer pH as well as other additives such as PEG is apparent to those skilled in the art and may result in similar crystals.

X-ray Crystallographic Analysis

Each of the constituent amino acids of S. aureus FemA is defined by a set of structure coordinates as set forth in Table 1. The term “structure coordinates” refers to Cartesian coordinates derived from mathematical equations related to the patterns obtained on diffraction of a monochromatic beam of x-rays by the atoms (scattering centers) of an S. aureus FemA complex in crystal form. The diffraction data are used to calculate an electron density map of the repeating unit of the crystal. The electron density maps are then used to establish the positions of the individual atoms of the S. aureus FemA protein or protein/ligand complex.

Slight variations in structure coordinates can be generated by mathematically manipulating the S. aureus FemA or S. aureus FemA/ligand structure coordinates. For example, the structure coordinates set forth in Table 1 could be manipulated by crystallographic permutations of the structure coordinates, fractionalization of the structure coordinates, integer additions or subtractions to sets of the structure coordinates, inversion of the structure coordinates or any combination of the above. Alternatively, modifications in the crystal structure due to mutations, additions, substitutions, and/or deletions of amino acids, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be structurally equivalent. Structural equivalence is described in more detail below.

It should be noted that slight variations in individual structure coordinates of the S. aureus FemA or S. aureus FemA/ligand complex, as defined above, would not be expected to significantly alter the nature of ligands that could associate with the substrate binding surfaces or binding sites. Thus, for example, a ligand that bound to the binding site of S. aureus FemA would also be expected to bind to or interfere with a “structurally equivalent” substrate binding surface or binding site.

For the purpose of this invention, any molecule or molecular complex or substrate binding surface or binding site thereof, or any portion thereof, that has a root mean square deviation of conserved residue backbone atoms (N, C_{α}, C, O) of less than about 1.5 Å, when superimposed on the relevant backbone atoms described by the reference structure coordinates listed in Table 1, is considered “structurally equivalent” to the reference molecule. That is to say, the crystal structures of those portions of the two molecules are substantially identical, within acceptable error. Particularly preferred structurally equivalent molecules or molecular complexes are those that are defined by the entire set of structure coordinates in Table 1, ±a root mean square deviation from the conserved backbone atoms of those amino acids of not more than 1.5 Å. More preferably, the root mean square deviation is less than about 1.0 Å.

The term “root mean square deviation” means the square root of the arithmetic mean of the squares of the deviations. It is a way to express the deviation or variation from a trend or object. For purposes of this invention, the “root mean square deviation” defines the variation in the backbone of a protein from the backbone of S. aureus FemA or a substrate binding surface or binding site portion thereof, as defined by the structure coordinates of S. aureus FemA described herein.

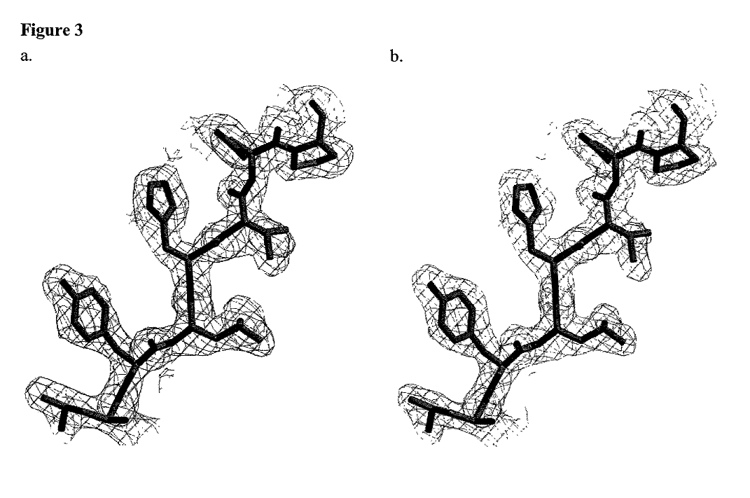

The structure of selenomethionine S. aureus Fem A was solved to 2. 1 Å by multiple anomalous dispersion (MAD) from selenomethionine incorporated protein. Data was collected at the IMCA beamline (17-ID) at the Advanced Photon Source (Argonne, Ill.) on a MarCCD detector (Table 5). Anomalous and dispersive difference Patterson maps revealed the presence of six selenium sites. A seventh site was identified by cross difference Fourier methods. The electron density map derived from the MAD phases showed the main chain and side chains of the protein with exceptional clarity (FIG. 3) and permitted rapid model building.

TABLE 5
Data collection and phasing statistics for data set collected on MarCCD detector on beamline
17-BM with an exposure time of 4 sec and a frame width of 1.0° .

| | λ0.96863 Å | λ0.97949 Å | λ0.97923 Å |
|---|---|---|---|
| | (12800 eV, remote) | (12659.8 eV, edge) | (12661.5 eV, peak) |
| Resolution | 2.10 Å | 2.06 Å | 2.09 Å |
| No. observations | 236,796 | 245,838 | 244,062 |
| No. unique refl. | 32,411 | 33,970 | 32,786 |
| % completeness | 99.7% | 98.7% | 99.1% |
| R_{sym} | 0.047 | 0.056 | 0.051 |
| R_{cullis} acentrics | — | 0.90 (0.52/1.00)* | 0.90 (0.50/1.00)* |
| R_{cullis} anomalous | — | 0.69 (0.28/0.97)* | 0.70 (0.25/0.97)* |
| Phasing power | | | |
| centrics | — | 0.59 (2.13/0.11)* | 0.55 (2.13/0.13)* |
| acentrics | — | 0.59 (2.49/0.17)* | 0.57 (2.67/0.18)* |
| Mean figure of merit | | | |
| (to 2.1 Å resolution) | | | |
| before solvent flattening | | 0.41 | |
| after solvent flattening | | 0.73 | |

*Data in parenthesis are for the lowest/highest resolution shells (20-6.8Å/2.33-2.10Å) respectively

Overview of the structure

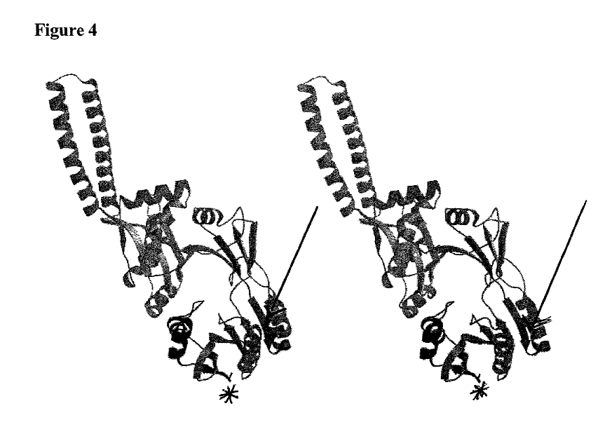

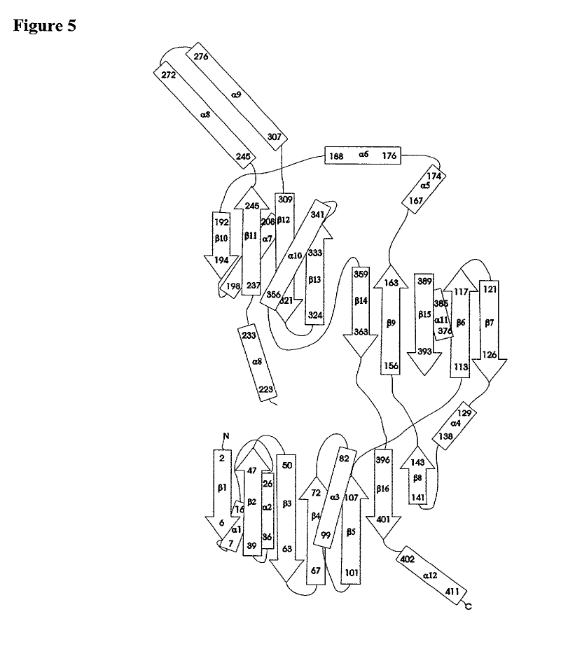

The structure contains two helical arms extending from a globular domain (FIG. 4). The helical arm is 48 Å long and 13 Å wide. The globular domain has approximate dimensions of 46 Å by 58 Å by 31 Å. FIG. 5 shows a secondary structure diagram for the S. aureus FemA structure. The globular domain of S. aureus FemA is a mixed α/β structure which is built upon to two twisted orthogonal β sheets which are surrounded by a number of α helices.

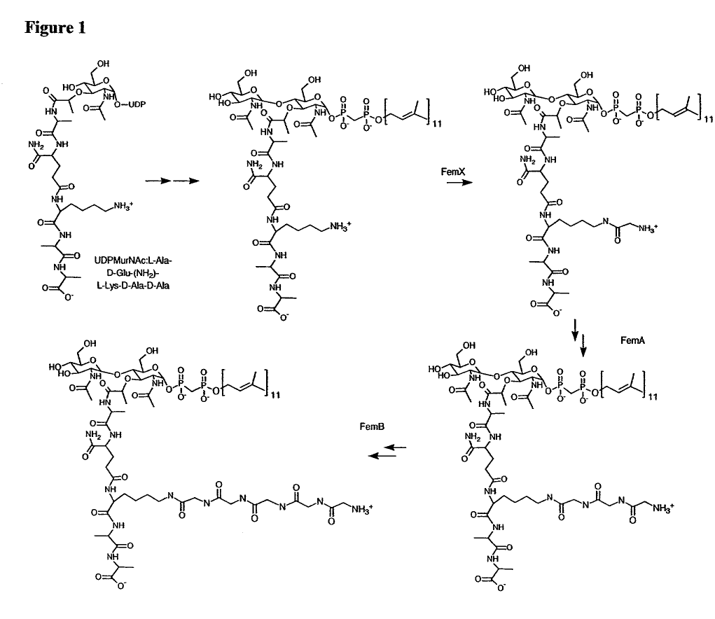

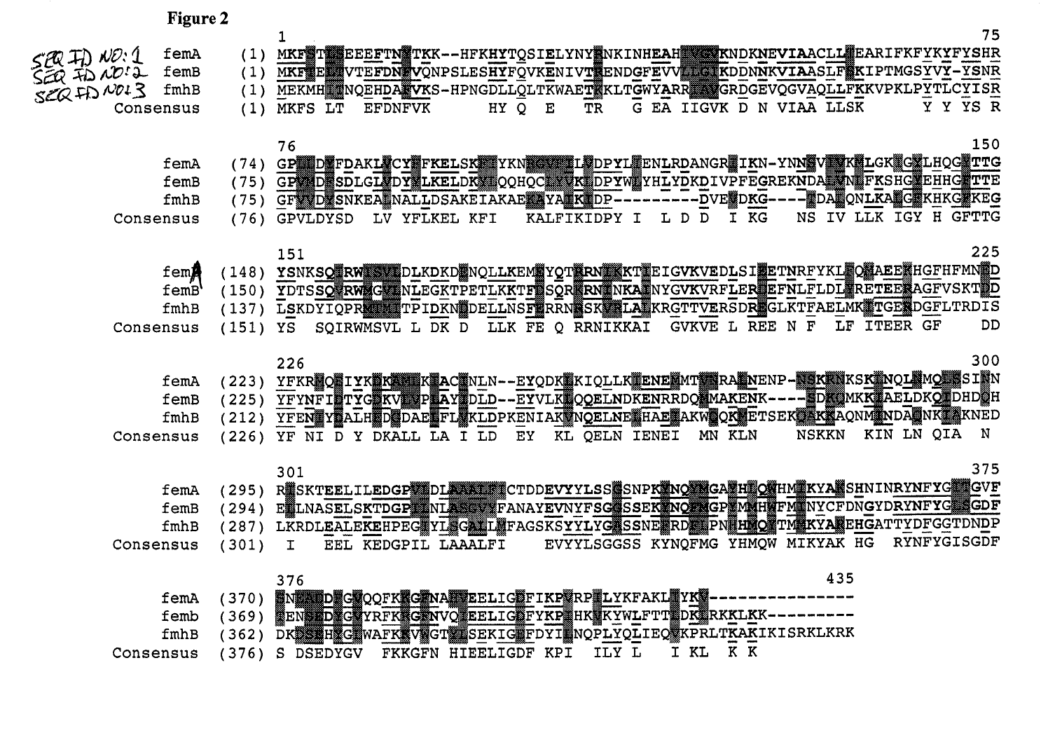

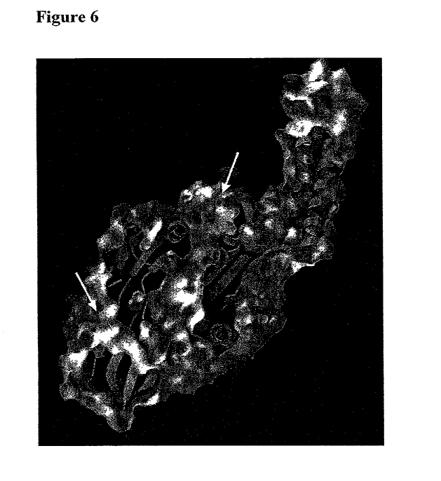

One of the first objectives in a structural study of an enzyme is to identify the binding sites for substrates and any potential active sites. Since the role for S. aureus FemA is to add glycines 2 and 3 in the pentaglycine bridge on the amino terminus of the lysine side chain, one of the substrates for FemA should be a disaccharide hexapeptide lipid macromolecule (FIG. 1). To bind this large molecule, a suitable binding surface should be present on the enzyme. Analysis of the binding surface for S. aureus FemA reveals a single long L-shaped channel on one face of the globular domain (FIG. 6). The residues which line this channel are listed in Table 2. Interestingly, the only break that occurs in the main chain due to disordered electron density in the entire S. aureus FemA structure is located over the top of this channel between α7 and α8. One could easily imagine that the residues between amino acids 208 and 223 are disordered because the disaccharide hexapeptide lipid macromolecule substrate is not present. Upon binding of this substrate, the disordered residues would close down during the catalytic step and then reopen after the addition of glycine is completed.

Related Structures Provide Clues to Mechanism: Binding of tRNA

Searching for related protein folds has become increasingly important as increasing numbers of protein structures are solved. This kind of analysis is particularly relevant for generating biochemical hypotheses for proteins of unknown or uncharacterized function. S. aureus FemA falls into the category of an enzyme with an uncharacterized function. While genetic evidence suggests its activity is to transfer glycine from tRNA^{Gly} to the peptidoglycan macromolecule, this biochemical activity has not been demonstrated in vitro primarily due to the complexity of the reagents. Therefore, analysis of the structure by searching for related folds can only create hypotheses which will need to be tested in a defined biochemical system. The entire Protein Data Bank was searched for folds related to S. aureus FemA using the program TOP (Lu, J. Appl. Cryst., 33:176-83 (2000)) searching with the helical arm domain and the globular domain separately.

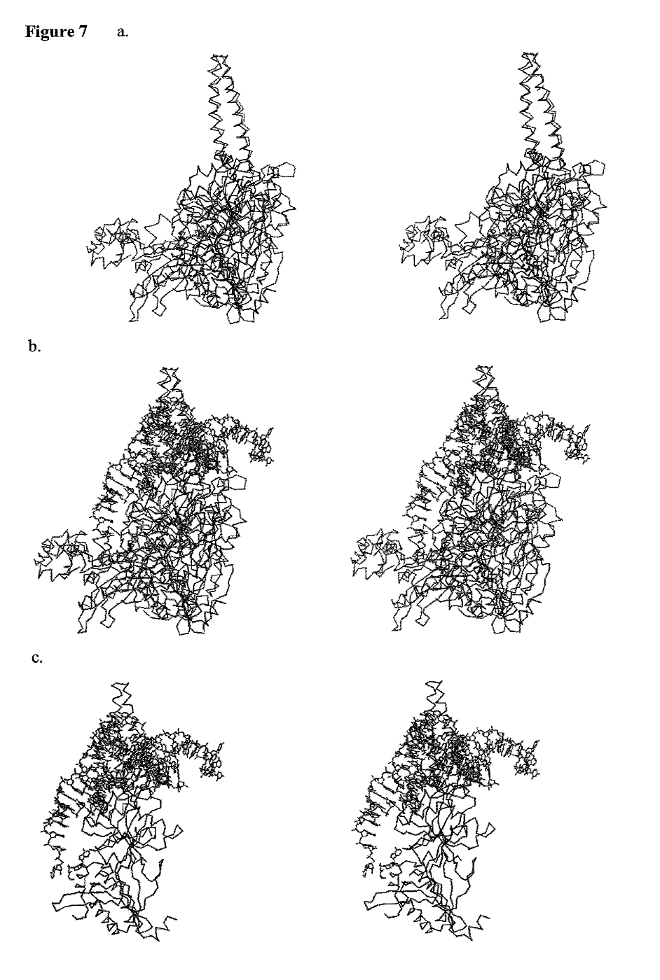

Helical arms similar to that seen in FemA (residues 246-307) have been observed in the structure of Thermus thermophilus seryl-tRNA-synthetase (Biou et al., Science, 263:1404-10 (1994); Cusack et al., EMBO J., 15:2834-42 (1996)) and other proteins that bind DNA or RNA. The general role of these helices is to provide a platform to interact with the polynucleotide. The presence of the helical arms suggest that these residues might serve to bind an amino acid-charged tRNA molecule. The glycines in the S. aureus cell wall have been shown to originate from charged tRNA molecules (Kamiryo et al., Biochem. Biophys. Res. Comm., 36:215-22 (1969); Kamiryo et al., J. Biol. Chem., 247:6306-11 (1972); Matsuhashi et al., J. Biol. Chem., 242:3191-3206 (1967); Thorndike et al., Biochem. Biophys. Res. Comm., 35:642-47 (1969)) though no in vitro biochemical assay for FemA has demonstrated this activity. The structure of FemA provides the first direct evidence that FemA is involved in the transfer of glycines from tRNA^{Gly} to the peptidoglcyan. The helical arm in FemA could provide a binding site for the glycyl-tRNA^{Gly} while it is transferring the glycine to the substrate. A model based on the structure of Thermus thermophilus seryl-tRNA-synthetase with a tRNA (Cusack et al., EMBO J., 15:2834-42 (1996)) shows that the CCA-Ser of the tRNA approaches subdomain 1A (see below) of FemA on the face opposite from the peptidoglycan L-channel (FIG. 7). It is also certainly possible that the glycyl-tRNA^{Gly} could be oriented in another direction in order to interact with the other face of FemA.

Related Structures Provide Clues to Mechanism: Binding of Other Substrates

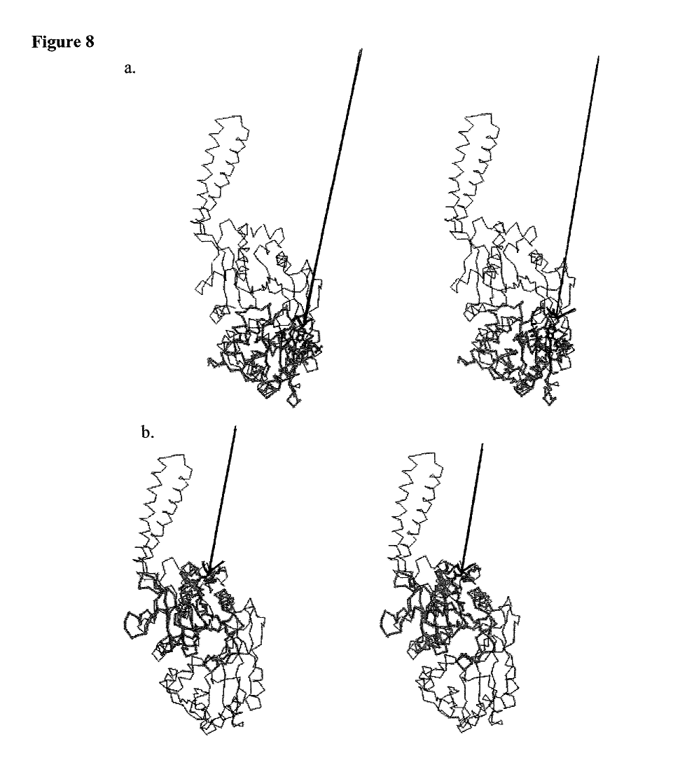

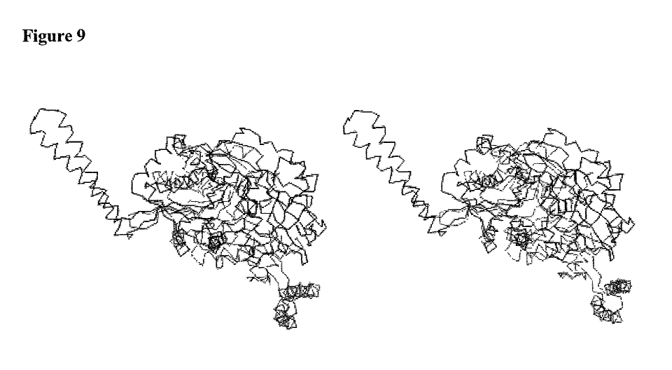

Searching the Protein Data Bank for protein folds that are related to the globular domain revealed two subdomains which are structurally homologous to the histone acetyltransferase domain of GCN5 and its homologs (Angus-Hill et al., J. Mol. Biol., 294:1311-25 (1999); Dutnall et al., Cell, 94:427-38 (1998); Rojas et al., Nature, 401:93-98 (1999); Trievel et al., Proc. Nat. Acad. Sci. USA, 96:8931-36 (1999); Wolf et al., Cell, 94:439-49 (1998); Wybenga-Groot et al., Structure Fold. Des., 7:497-507 (1999); Elements et al., EMBO J., 18:3521-32 (1999)). The database searching revealed that there are two subdomains within the globular portion of FemA-domain 1A, residues 1-108 and 396-411, and domain 1B, residues 113-395. Within each of these subdomains, a GCN5 N-acetyltransferase related fold can be found by superposition of Tetrahymena GCN5 with the appropriate residues in S. aureus FemA (FIG. 8). Residues that are found near the coenzyme A binding site in the GCN5 fold for domains 1A and 1B are listed in Tables 3 and 4. In addition, a superposition of domain 1A with domain 1B from S. aureus FemA shows the duplication of this fold within the protein (FIG. 9).

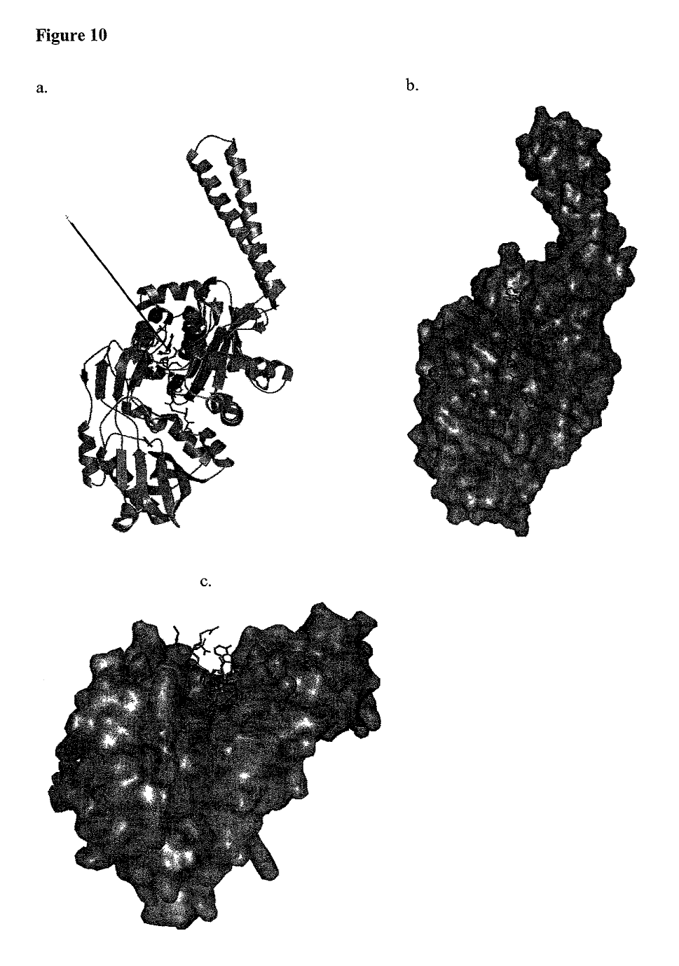

The coenzyme A binding sites within each subdomain of FemA appear reasonable places for a small molecule to bind to the enzyme. Interestingly, GCN5 also binds a peptide which contains a lysine residue that is the site for acteylation (Rojas et al., Nature, 401:93-98 (1999)). Localization of this GCN5 N-acetyltransferase peptide binding site on each of the FemA subdomains indicates that the surface on domain 1A is buried within the protein, whereas the surface on domain 1B corresponds to part of the L-shaped channel on S. aureus FemA (FIG. 10). This observation provides further evidence that the L-shaped channel is the correct binding site for the disaccharide pentapeptide substrate.

Models for FemA Function

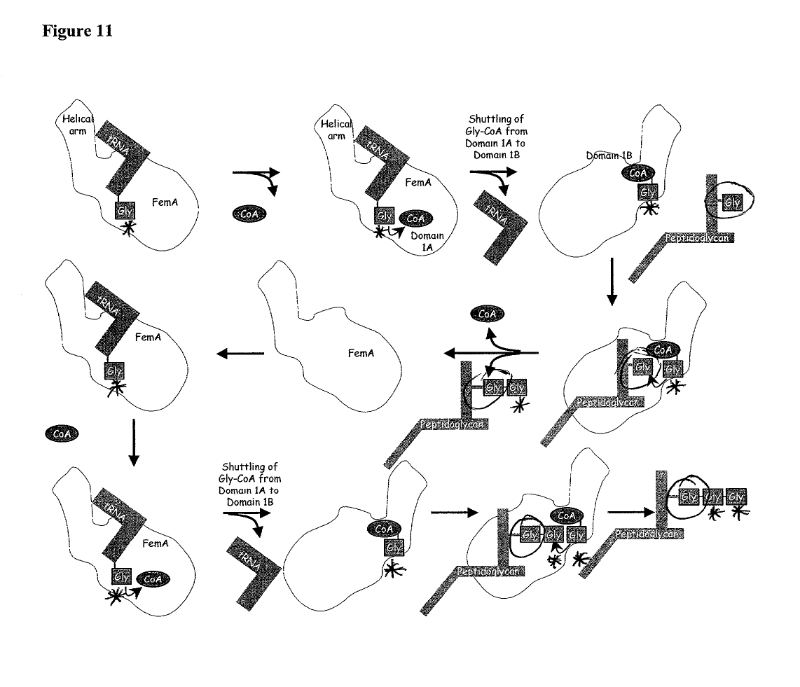

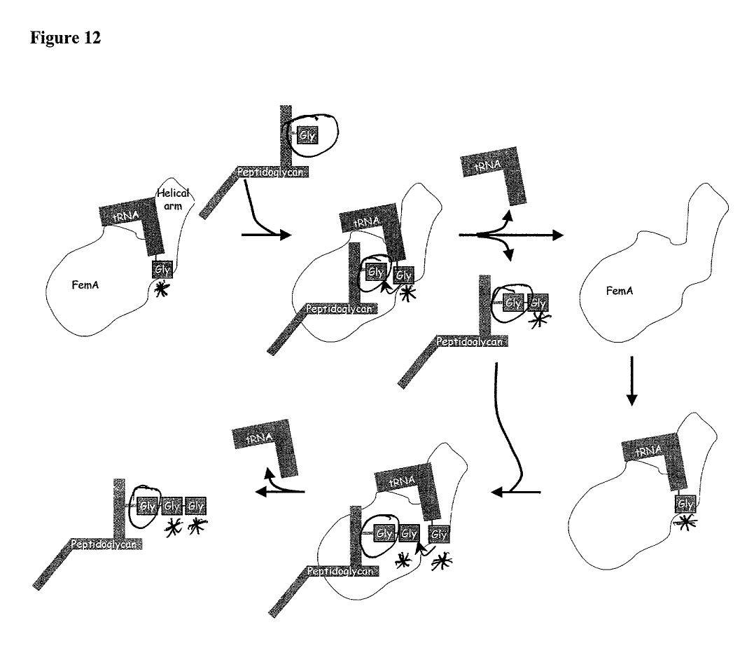

Structural folds often give clues for the function and mechanism of novel proteins that might not be determined by sequence comparison (Terwilliger et al., Pro. Sci., 7:1851-56 (1998)). Analysis of the structure of S. aureus FemA and comparison between its structure and related structures in the Protein Data Bank provide the basis for two potential models for FemA function (FIGS. 11 and 12). In both models, FemA transfers a glycine to the amino terminus of the single glycine attached to the ε-amino group of the pentapeptide lysine by FemX (FmhB). The binding site for this substrate coincides with the L-shaped channel found on one face of the globular domain as shown in FIG. 6. The additions of two glycines by FemA is likely in a sequential fashion.

The “Coenzyme A Shuttle” model (FIG. 11) provides for transfer of the glycine residue via a coenzyme A carrier molecule. Binding of glycyl-tRNA^{GlY} is shown first, though there is no direct evidence for this. Transfer of the glycine from the glycyl-tRNA^{GlY} to a molecule of coenzyme A occurs on subdomain 1A. Subsequently, the charged coenzyme A-glycine is shuttled to subdomain 1B for transfer to the peptidoglycan substrate. Amino-acyl-coenzyme A molecules have been shown to be involved in nonribosomal peptide synthesis (Belshaw et al., Science, 284:486-89 (1999); Marahiel et al., Chem. Rev., 97:2651-73 (1997)) and, in fact, transfer of amino acids from charged tRNAs to coenzyme A has been demonstrated (Jakubowski, Biochemistry, 37:5147-53 (1998)). Subsequent to the shuttling of the amino-acyl-conenzyme A molecule, transfer of the glycine would occur in subdomain 1B to the N terminus of the glycine already attached to the ε-amino group of the pentapeptide lysine. The addition of the second glycine is shown to occur in the same manner as the first addition. At the present time, it is unclear whether the intermediate substrate (lipid-disaccharide-pentapeptide-Gly-Gly) is actually released from FemA before the addition of the third glycine.

A second model, the “tRNA Transfer” model, that would not require the involvement of coenzyme A is shown in FIG. 11. In this model, direct transfer of the glycine from the glycyl-tRNA^{Gly} to the peptidoglycan would be similar to peptide bond formation in ribosomal peptide biosynthesis. Attack of the free amino group from the Gly-Lys-pentapeptide on the Gly-tRNA^{Gly} would extend the glycine chain by one residue. A subsequent round of glycyl-tRNA^{Gly} binding and transfer would account for the addition of the second glycine.

Binding Surfaces/Binding Sites/Other Structural Features

The present invention has provided, for the first time, information about the shape and structure of the substrate binding surface and binding sites of S. aureus FemA.

Substrate binding surfaces and binding sites are of significant utility in fields such as drug discovery. The association of natural ligands or substrates with the substrate binding surfaces or binding sites of their corresponding receptors or enzymes is the basis of many biological mechanisms of action. Similarly, many drugs exert their biological effects through association with the substrate binding surfaces or binding sites of receptors and enzymes. Such associations may occur with all or any parts of the substrate binding surfaces or binding sites. An understanding of such associations helps lead to the design of drugs having more favorable associations with their target, and thus improved biological effects. Therefore, this information is valuable in designing potential modifiers of S. aureus FemA-like activity, as discussed in more detail below.

The term “substrate binding surface or binding site,” as used herein, refers to a region of a molecule or molecular complex, that, as a result of its shape, favorably associates with another chemical entity or compound. Thus, a substrate binding surface or binding site may include or consist of features such as interfaces between domains. Chemical entities or compounds that may associate with a substrate binding surface or binding site include, but are not limited to, cofactors, substrates, inhibitors, agonists, antagonists, etc.

The substrate binding surface of S. aureus FemA preferably includes at least a portion of the amino acids listed in Table 2, as shown in Table 1. A binding site of S. aureus FemA preferably includes at least a portion of the amino acids listed in Table 2 or 3, as shown in Table 1. As used herein, “at least a portion of the amino acids” means at least about 50% of the amino acids, preferably at least about 70% of the amino acids, more preferably at least about 90% of the amino acids, and most preferably all the amino aicds. It will be readily apparent to those of skill in the art that the numbering of amino acids in other isoforms of S. aureus FemA may be different.

The amino acid constituents of an S. aureus FemA substrate binding surface or binding site as defined herein, as well as selected constituent atoms thereof, are positioned in three dimensions in accordance with the structure coordinates listed in Table 1. In one aspect, the structure coordinates defining the substrate binding surface or binding site of S. aureus FemA include structure coordinates of all atoms in the constituent amino acids; in another aspect, the structure coordinates of the substrate binding surface or binding site include structure coordinates of just the backbone atoms of the constituent atoms.

The term “S. aureus FemA-like substrate binding surface or binding site” refers to a portion of a molecule or molecular complex whose shape is sufficiently similar to at least a portion of the substrate binding surface or binding site of S. aureus FemA as to be expected to bind common structurally or functionally related ligands. A structurally equivalent substrate binding surface or binding site is defined by a root mean square deviation from the structure coordinates of the backbone atoms of the amino acids that make up the substrate binding surfaces or binding sites in S. aureus FemA (as set forth in Table 1) of at most about 1.5 Å. How this calculation is obtained is described below.

The term “associating with” refers to a condition of proximity between a chemical entity or compound, or portions thereof, and an S. aureus FemA molecule or portions thereof. The association may be non-covalent, wherein the juxtaposition is energetically favored by hydrogen bonding, van der Waals forces, or electrostatic interactions, or it may be covalent.

Accordingly, the invention thus provides molecules or molecular complexes including an S. aureus FemA substrate binding surfaces or binding sites or S. aureus FemA-like substrate binding surfaces or binding sites, as defined by the sets of structure coordinates described above.

Three-Dimensional Configurations

The structure coordinates generated for S. aureus FemA or the S. aureus FemA/ligand complex or one of its substrate binding surfaces or binding sites shown in Table 1 define a unique configuration of points in space. Those of skill in the art understand that a set of structure coordinates for protein or an protein/ligand complex, or a portion thereof, define a relative set of points that, in turn, define a configuration in three dimensions. A similar or identical configuration can be defined by an entirely different set of coordinates, provided the distances and angles between coordinates remain essentially the same. In addition, a scalable configuration of points can be defined by increasing or decreasing the distances between coordinates by a scalar factor while keeping the angles essentially the same.

The present invention thus includes the three-dimensional configuration of points derived from the structure coordinates of at least a portion of an S. aureus FemA molecule or molecular complex, as shown in Table 1, as well as structurally equivalent configurations, as described below. Preferably, the three-dimensional configuration includes points derived from structure coordinates representing the locations of a plurality of the amino acids defining the S. aureus FemA substrate binding surface or binding site. In one embodiment, the three-dimensional configuration includes points derived from structure coordinates representing the locations of the backbone atoms of a plurality of amino acids defining the S. aureus FemA substrate binding surface, preferably as listed in Table 2; in another embodiment, the three-dimensional configuration includes points derived from structure coordinates representing the locations the backbone atoms of a plurality of amino acids defining an S. aureus FemA binding site, preferably as listed in Table 3 or 4; in another embodiment, the three-dimensional configuration includes points derived from structure coordinates representing the locations of the side chain and the backbone atoms (other than hydrogens) of a plurality of the amino acids defining the S. aureus FemA substrate binding surface, preferably as listed in Table 2; and in another embodiment, the three-dimensional configuration includes points derived from structure coordinates representing the locations of the side chain and the backbone atoms (other than hydrogens) of a plurality of the amino acids defining an S. aureus FemA binding site, preferably as listed in Table 3 or 4. In yet another embodiment, the three-dimensional configuration includes points derived from structure coordinates representing the locations the backbone atoms of at least 40 amino acids that are contiguous in the amino acid sequence of S. aureus FemA (SEQ ID NO: 1). In still another embodiment, the three-dimensional configuration includes points derived from structure coordinates representing the locations the side chain atoms and the backbone atoms of at least 40 amino acids that are contiguous in the amino acid sequence of S. aureus FemA (SEQ ID NO: 1).

Likewise, the invention also includes the three-dimensional configuration of points derived from structure coordinates of molecules or molecular complexes that are structurally homologous to S. aureus FemA, as well as structurally equivalent configurations. Structurally homologous molecules or molecular complexes are defined below. Advantageously, structurally homologous molecules can be identified using the structure coordinates of S. aureus FemA (Table 1) according to a method of the invention.

The configurations of points in space derived from structure coordinates according to the invention can be visualized as, for example, a holographic image, a stereodiagram, a model or a computer-displayed image, and the invention thus includes such images, diagrams or models.

Structurally Equivalent Crystal Structures

Various computational analyses can be used to determine whether a molecule or the substrate binding surface or binding site portion thereof is “structurally equivalent,” defined in terms of its three-dimensional structure, to all or part of S. aureus FemA or its substrate binding surface or binding site. Such analyses may be carried out in current software applications, such as the Molecular Similarity application of QUANTA (Molecular Simulations Inc., San Diego, CA) version 4. 1, and as described in the accompanying User's Guide.

The Molecular Similarity application permits comparisons between different structures, different conformations of the same structure, and different parts of the same structure. The procedure used in Molecular Similarity to compare structures is divided into four steps: (1) load the structures to be compared; (2) define the atom equivalences in these structures; (3) perform a fitting operation; and (4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention equivalent atoms are defined as protein backbone atoms (N, Cα, C, and O) for all conserved residues between the two structures being compared. A conserved residue is defined as a residue that is structurally or functionally equivalent. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

Machine Readable Storage Media

Transformation of the structure coordinates for all or a portion of S. aureus FemA or the S. aureus FemA/ligand complex or one of its substrate binding surfaces or binding sites, for structurally homologous molecules as defined below, or for the structural equivalents of any of these molecules or molecular complexes as defined above, into three-dimensional graphical representations of the molecule or complex can be conveniently achieved through the use of commercially-available software.

The invention thus further provides a machine-readable storage medium including a data storage material encoded with machine readable data which, when using a machine programmed with instructions for using said data, displays a graphical three-dimensional representation of any of the molecule or molecular complexes of this invention that have been described above. In a preferred embodiment, the machine-readable data storage medium includes a data storage material encoded with machine readable data which, when using a machine programmed with instructions for using said data, displays a graphical three-dimensional representation of a molecule or molecular complex including all or any parts of an S. aureus FemA substrate binding surface or binding site or an S. aureus FemA-like substrate binding surface or binding site, as defined above. In another preferred embodiment, the machine-readable data storage medium displays a graphical three-dimensional representation of a molecule or molecular complex defined by the structure coordinates of all of the amino acids in Table 1, +a root mean square deviation from the backbone atoms of said amino acids of not more than 1.5 Å.

In an alternative embodiment, the machine-readable data storage medium includes a data storage material encoded with a first set of machine readable data which includes the Fourier transform of the structure coordinates set forth in Table 1, and which, when using a machine programmed with instructions for using said data, can be combined with a second set of machine readable data including the x-ray diffraction pattern of a molecule or molecular complex to determine at least a portion of the structure coordinates corresponding to the second set of machine readable data.

For example, a system for reading a data storage medium may include a computer including a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid cyrstal displays (“LCDs”), electroluminescent displays, vacuum fluorescent displays, field emission displays (“FEDs”), plasma displays, projection panels, etc.), one or more user input devices (e.g., keyboards, microphones, mice, touch screens, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this invention may be inputted via the use of a modem or modems connected by a telephone line or dedicated data line. Alternatively or additionally, the input hardware may include CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of a substrate binding surface or binding site of this invention using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this invention. Such programs are discussed in reference to the computational methods of drug discovery as described herein. References to components of the hardware system are included as appropriate throughout the following description of the data storage medium.

Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

Structurally Homologous Molecules, Molecular Complexes, And Crystal Structures

The structure coordinates set forth in Table 1 can be used to aid in obtaining structural information about another crystallized molecule or molecular complex. A “molecular complex” means a protein in covalent or non-covalent association with a chemical entity or compound. The method of the invention allows determination of at least a portion of the three-dimensional structure of molecules or molecular complexes which contain one or more structural features that are similar to structural features of S. aureus FemA. These molecules are referred to herein as “structurally homologous” to S. aureus FemA. Similar structural features can include, for example, regions of amino acid identity, conserved active site or binding site motifs, and similarly arranged secondary structural elements (e.g., α helices and β sheets). Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Preferably, two amino acid sequences are compared using the Blastp program, version 2.0.9, of the BLAST 2 search algorithm, as described by Tatusova et al., FEMS Microbiol Lett., 174:247-50 (1999), and available at http://www.ncbi.nlm.nih.gov/gorf/bl2.html. Preferably, the default values for all BLAST 2 search parameters are used, including matrix=BLOSUM62; open gap penalty=11, extension gap penalty=1, gap x dropoff=50, expect=10, wordsize=3, and filter on. In the comparison of two amino acid sequences using the BLAST search algorithm, structural similarity is referred to as “identity.” Preferably, a structurally homologous molecule is a protein that has an amino acid sequence sharing at least 65% identity with the amino acid sequence of S. aureus FemA (SEQ ID NO: 1). More preferably, a protein that is structurally homologous to S. aureus FemA includes at least one contiguous stretch of at least 50 amino acids that shares at least 80% amino acid sequence identity with the analogous portion of S. aureus FemA. Methods for generating structural information about the structurally homologous molecule or molecular complex are well-known and include, for example, molecular replacement techniques.

Therefore, in another embodiment this invention provides a method of utilizing molecular replacement to obtain structural information about a molecule or molecular complex whose structure is unknown including the steps of:

(a) crystallizing the molecule or molecular complex of unknown structure;

(b) generating an x-ray diffraction pattern from said crystallized molecule or molecular complex; and

(c) applying at least a portion of the structure coordinates set forth in Table 1 to the x-ray diffraction pattern to generate a three-dimensional electron density map of the molecule or molecular complex whose structure is unknown.

By using molecular replacement, all or part of the structure coordinates of S. aureus FemA or the S. aureus FemA/ligand complex as provided by this invention (and set forth in Table 1) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a structurally homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of S. aureus FemA or the S. aureus FemA/ligand complex according to Table 1 within the unit cell of the crystal of the unknown molecule or molecular complex so as best to account for the observed x-ray diffraction pattern of the crystal of the molecule or molecular complex whose structure is unknown. Phases can then be calculated from this model and combined with the observed x-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex (E. Lattman, “Use of the Rotation and Translation Functions,” in Meth. Enzymol., 115:55-77 (1985); M. G. Rossman, ed., “The Molecular Replacement Method,” Int. Sci. Rev. Ser., No. 13, Gordon & Breach, New York (1972)).

Structural information about a portion of any crystallized molecule or molecular complex that is sufficiently structurally homologous to a portion of S. aureus FemA can be resolved by this method. In addition to a molecule that shares one or more structural features with S. aureus FemA as described above, a molecule that has similar bioactivity, such as the same catalytic activity, substrate specificity or ligand binding activity as S. aureus FemA, may also be sufficiently structurally homologous to S. aureus FemA to permit use of the structure coordinates of S. aureus FemA to solve its crystal structure.

In a preferred embodiment, the method of molecular replacement is utilized to obtain structural information about a molecule or molecular complex, wherein the molecule or molecular complex includes at least one S. aureus FemA subunit or homolog. A “subunit” of S. aureus FemA is an S. aureus FemA molecule that has been truncated at the N-terminus or the C-terminus, or both. In the context of the present invention, a “homolog” of S. aureus FemA is a protein that contains one or more amino acid substitutions, deletions, additions, or rearrangements with respect to the amino acid sequence of S. aureus FemA, but that, when folded into its native conformation, exhibits or is reasonably expected to exhibit at least a portion of the tertiary (three-dimensional) structure of S. aureus FemA. For example, structurally homologous molecules can contain deletions or additions of one or more contiguous or noncontiguous amino acids, such as a loop or a domain. Structurally homologous molecules also include “modified” S. aureus FemA molecules that have been chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

A heavy atom derivative of S. aureus FemA is also included as an S. aureus FemA homolog. The term “heavy atom derivative” refers to derivatives of S. aureus FemA produced by chemically modifying a crystal of S. aureus FemA. In practice, a crystal is soaked in a solution containing heavy metal atom salts, or organometallic compounds, e.g., lead chloride, gold thiomalate, thiomersal or uranyl acetate, which can diffuse through the crystal and bind to the surface of the protein. The location(s) of the bound heavy metal atom(s) can be determined by x-ray diffraction analysis of the soaked crystal. This information, in turn, is used to generate the phase information used to construct three-dimensional structure of the protein (T. L. Blundell and N. L. Johnson, Protein Crystallography, Academic Press (1976)).

Because S. aureus FemA can crystallize in more than one crystal form, the structure coordinates of S. aureus FemA as provided by this invention are particularly useful in solving the structure of other crystal forms of S. aureus FemA or S. aureus FemA complexes.

The structure coordinates of S. aureus FemA in Table 1 are also particularly useful to solve the structure of crystals of S. aureus FemA, S. aureus FemA mutants or S. aureus FemA homologs co-complexed with a variety of chemical entities. This approach enables the determination of the optimal sites for interaction between chemical entities, including candidate S. aureus FemA modifiers and S. aureus FemA. Potential sites for modification within the various binding site of the molecule can also be identified. This information provides an additional tool for determining the most efficient binding interactions, for example, increased hydrophobic interactions, between S. aureus FemA and a chemical entity. For example, high resolution x-ray diffraction data collected from crystals exposed to different types of solvent allows the determination of where each type of solvent molecule resides. Small molecules that bind tightly to those sites can then be designed and synthesized and tested for their potential modification of S. aureus FemA.

All of the complexes referred to above may be studied using well-known x-ray diffraction techniques and may be refined versus 1.5-3 Å resolution x-ray data to an R value of about 0.20 or less using computer software, such as X-PLOR (Yale University, (1992), distributed by Molecular Simulations, Inc.; see, e.g., Blundell & Johnson, supra; Meth. Enzymol., Vol. 114 & 115, H. W. Wyckoff et al., eds., Academic Press (1985)). This information may thus be used to optimize known modifiers of S. aureus FemA activity, and more importantly, to design new modifiers of S. aureus FemA activity.

The invention also includes the unique three-dimensional configuration defined by a set of points defined by the structure coordinates for a molecule or molecular complex structurally homologous to S. aureus FemA, S. aureus FemA binding sites, and/or S. aureus FemA substrate binding surfaces, as determined using the method of the present invention, structurally equivalent configurations, and magnetic storage media including such set of structure coordinates.

Further, the invention includes structurally homologous molecules as identified using the method of the invention.

Homology Modeling

Using homology modeling, a computer model of an S. aureus FemA homolog can be built or refined without crystallizing the homolog. First, a preliminary model of the S. aureus FemA homolog is created by sequence alignment with S. aureus FemA, secondary structure prediction, the screening of structural libraries, or any combination of those techniques. Computational software may be used to carry out the sequence alignments and the secondary structure predictions. Structural incoherences, e.g., structural fragments around insertions and deletions, can be modeled by screening a structural library for peptides of the desired length and with a suitable conformation. For prediction of the side chain conformation, a side chain rotamer library may be employed. Where the S. aureus FemA homolog has been crystallized, the final homology model can be used to solve the crystal structure of the homolog by molecular replacement, as described above. Next, the preliminary model is subjected to energy minimization to yield an energy minimized model. The energy minimized model may contain regions where stereochemistry restraints are violated, in which case such regions are remodeled to obtain a final homology model. The homology model is positioned according to the results of molecular replacement, and subjected to further refinement including molecular dynamics calculations.

Rational Drug Design

Computational techniques can be used to screen, identify, select and design chemical entities capable of associating with S. aureus FemA or structurally homologous molecules. Knowledge of the structure coordinates for S. aureus FemA permits the design and/or identification of synthetic compounds and/or other molecules which have a shape complementary to the conformation of the S. aureus FemA binding site. In particular, computational techniques can be used to identify or design chemical entities that are potential modifiers of S. aureus FemA activity, such as inhibitors, agonists and antagonists, that associate with an S. aureus FemA substrate binding surface or binding site or an S. aureus FemA-like substrate binding surface or binding site. Potential modifiers may bind to or interfere with all or a portion of the active site of S. aureus FemA, and can be competitive, non-competitive, or uncompetitive inhibitors; or interfere with dimerization by binding at the interface between the two monomers. Once identified and screened for biological activity, these inhibitors/agonists/antagonists may be used therapeutically or prophylactically to block S. aureus FemA activity and, thus, results in inhibition of growth or death of bacteria. Structure-activity data for analogs of ligands that bind to or interfere with S. aureus FemA or S. aureus FemA-like substrate binding surfaces or binding sites can also be obtained computationally.

The term “chemical entity,” as used herein, refers to chemical compounds, complexes of two or more chemical compounds, and fragments of such compounds or complexes. Chemical entities that are determined to associate with S. aureus FemA are potential drug candidates. Data stored in a machine-readable storage medium that displays a graphical three-dimensional representation of the structure of S. aureus FemA or a structurally homologous molecule, as identified herein, or portions thereof may thus be advantageously used for drug discovery. The structure coordinates of the chemical entity are used to generate a three-dimensional image that can be computationally fit to the three-dimensional image of S. aureus FemA or a structurally homologous molecule. The three-dimensional molecular structure encoded by the data in the data storage medium can then be computationally evaluated for its ability to associate with chemical entities. When the molecular structures encoded by the data is displayed in a graphical three-dimensional representation on a computer screen, the protein structure can also be visually inspected for potential association with chemical entities.

One embodiment of the method of drug design involves evaluating the degree of association of a known chemical entity with S. aureus FemA or a structurally homologous molecule, particularly with an S. aureus FemA substrate binding surface or binding site or S. aureus FemA-like substrate binding surface or binding site. The method of drug design thus includes computationally evaluating the potential of a selected chemical entity to associate with any of the molecules or molecular complexes set forth above. This method includes the steps of: (a) employing computational means to perform a fitting operation between the selected chemical entity and a substrate binding surface or binding site of the molecule or molecular complex; and (b) analyzing the results of said fitting operation to quantify the association between the chemical entity and the substrate binding surface or binding site.

In another embodiment, the method of drug design involves computer-assisted design of chemical entities that associate with S. aureus FemA, its homologs, or portions thereof. Chemical entities can be designed in a step-wise fashion, one fragment at a time, or may be designed as a whole or “de novo.”

To be a viable drug candidate, the chemical entity identified or designed according to the method must be capable of structurally associating with at least part of an S. aureus FemA or S. aureus FemA-like substrate binding surfaces or binding sites, and must be able, sterically and energetically, to assume a conformation that allows it to associate with the S. aureus FemA or S. aureus FemA-like substrate binding surface or binding site. Non-covalent molecular interactions important in this association include hydrogen bonding, van der Waals interactions, hydrophobic interactions, and electrostatic interactions. Conformational considerations include the overall three-dimensional structure and orientation of the chemical entity in relation to the substrate binding surface or binding site, and the spacing between various functional groups of an entity that directly interact with the S. aureus FemA-like substrate binding surface or binding site or homologs thereof.

Optionally, the degree of binding of a chemical entity to an S. aureus FemA or S. aureus FemA-like substrate binding surface or binding site is analyzed using computer modeling techniques prior to the actual synthesis and testing of the chemical entity. If these computational experiments suggest insufficient interaction and association between it and the S. aureus FemA or S. aureus FemA-like substrate binding surface or binding site, testing of the entity is obviated. However, if computer modeling indicates a strong interaction, the molecule may then be synthesized and tested for its ability to bind to or interfere with an S. aureus FemA or S. aureus FemA-like substrate binding surface or binding site. In general, binding assays to determine if a compound actually binds to a protein can also be performed, and are often well known in the art. Binding assays may employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof.

One method for determining whether a modifier binds to a protein is isothermal denaturation. This method includes taking a sample of a protein (in the presence or absence of substrates) at a fixed elevated temperature where denaturation of the protein occurs in a given time frame, adding the chemical entity to the protein, and monitoring the rate of denaturation. If the chemical entity does bind to the protein, it is expected that the rate of denaturation would be slower in the presence of the chemical entity than in the absence of the chemical entity. For example, this method has been described in Epps et al., Anal. Biochem., 292:40-50 (2001).

One skilled in the art may use one of several methods to screen chemical entities or fragments for their ability to associate with an S. aureus FemA or S. aureus FemA-like substrate binding surface or binding site. This process may begin by visual inspection of, for example, an S. aureus FemA or S. aureus FemA-like substrate binding surface or binding site on the computer screen based on the S. aureus FemA structure coordinates in Table 1 or other coordinates which define a similar shape generated from the machine-readable storage medium. Selected fragments or chemical entities may then be positioned in a variety of orientations, or docked, within the substrate binding surface or binding site. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting fragments or chemical entities. Examples include GRID (P. J. Goodford, J. Med. Chem., 28:849-57 (1985); available from Oxford University, Oxford, UK); MCSS (A. Miranker et al., Proteins: Struct. Funct. Gen., 1 1:29-34 (1991); available from Molecular Simulations, San Diego, Calif.); AUTODOCK (D. S. Goodsell et al., Proteins: Struct. Funct. Genet., 8:195-202 (1990); available from Scripps Research Institute, La Jolla, Calif.); and DOCK (I.D. Kuntz et al., J. Mol. Biol., 161:269-88 (1982); available from University of California, San Francisco, Calif.).

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of S. aureus FemA. This would be followed by manual model building using software such as QUANTA or SYBYL (Tripos Associates, St. Louis, MO).

Useful programs to aid one of skill in the art in connecting the individual chemical entities or fragments include, without limitation, CAVEAT (P.A. Bartlett et al., in Molecular Recognition in Chemical and Biological Problems, Special Publ., Royal Chem. Soc., 78:182-96 (1989); G. Lauri et al., J. Comput. Aided Mol. Des., 8:51-66 (1994); available from the University of California, Berkeley, Calif.); 3D database systems such as ISIS (available from MD-L Information Systems, San Leandro, Calif.; reviewed in Y. C. Martin, J. Med. Chem. 35:2145-54 (1992)); and HOOK (M. B. Eisen et al., Proteins: Struc., Funct., Genet., 19:199-221 (1994); available from Molecular Simulations, San Diego, Calif.).

S. aureus FemA binding compounds may be designed “de novo” using either an empty binding site or optionally including some portion(s) of a known modifier(s). There are many de novo ligand design methods including, without limitation, LUDI (H. -J. Bohm, J. Comp. Aid. Molec. Design., 6:61-78 (1992); available from Molecular Simulations Inc., San Diego, Calif.); LEGEND (Y. Nishibata et al., Tetrahedron, 47:8985 (1991); available from Molecular Simulations Inc., San Diego, Calif.); LeapFrog (available from Tripos Associates, St. Louis, Mo.); and SPROUT (V. Gillet et al., J. Comput. Aided Mol. Design, 7:127-53 (1993); available from the University of Leeds, UK).

Once a compound has been designed or selected by the above methods, the efficiency with which that entity may bind to or interfere with an S. aureus FemA or S. aureus FemA-like substrate binding surface or binding site may be tested and optimized by computational evaluation. For example, an effective S. aureus FemA or S. aureus FemA-like substrate binding surface or binding site modifier must preferably demonstrate a relatively small difference in energy between its bound and free states (i.e., a small deformation energy of binding). Thus, the most efficient S. aureus FemA or S. aureus FemA-like substrate binding surface or binding site modifiers should preferably be designed with a deformation energy of binding of not greater than about 10 kcal/mole; more preferably, not greater than 7 kcal/mole. S. aureus FemA or S. aureus FemA-like substrate binding surface or binding site modifiers may interact with the substrate binding surface or binding site in more than one conformation that is similar in overall binding energy. In those cases, the deformation energy of binding is taken to be the difference between the energy of the free entity and the average energy of the conformations observed when the modifier binds to the protein.

An entity designed or selected as binding to or interfering with an S. aureus FemA or S. aureus FemA-like substrate binding surface or binding site may be further computationally optimized so that in its bound state it would preferably lack repulsive electrostatic interaction with the target enzyme and with the surrounding water molecules. Such non-complementary electrostatic interactions include repulsive charge-charge, dipole-dipole, and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. (1995)); AMBER, version 4.1 (P. A. Kollman, University of California at San Francisco, (1995)); QUANTA/CHARMM (Molecular Simulations, Inc., San Diego, Calif. (1995)); Insight II/Discover (Molecular Simulations, Inc., San Diego, Calif. (1995)); DelPhi (Molecular Simulations, Inc., San Diego, Calif. (1995)); and AMSOL (Quantum Chemistry Program Exchange, Indiana University). These programs may be implemented, for instance, using a Silicon Graphics workstation such as an Indigo² with “IMPACT” graphics. Other hardware systems and software packages will be known to those skilled in the art.

Another approach encompassed by this invention is the computational screening of small molecule databases for chemical entities or compounds that can bind in whole, or in part, to a S. aureus FemA or S. aureus FemA-like substrate binding surface or binding site. In this screening, the quality of fit of such entities to the binding site may be judged either by shape complementarity or by estimated interaction energy (E. C. Meng et al., J. Comp. Chem., 13:505-24 (1992)).

This invention also enables the development of chemical entities that can isomerize to short-lived reaction intermediates in the chemical reaction of a substrate or other compound that binds to or with S. aureus FemA. Time-dependent analysis of structural changes in S. aureus FemA during its interaction with other molecules is carried out. The reaction intermediates of S. aureus FemA can also be deduced from the reaction product in co-complex with S. aureus FemA. Such information is useful to design improved analogs of known modifiers of S. aureus FemA activity or to design novel classes of modifiers based on the reaction intermediates of the S. aureus FemA and modifier co-complex. This provides a novel route for designing S. aureus FemA modifiers with both high specificity and stability.

Yet another approach to rational drug design involves probing the S. aureus FemA crystal of the invention with molecules including a variety of different functional groups to determine optimal sites for interaction between candidate S. aureus FemA modifiers and the protein. For example, high resolution x-ray diffraction data collected from crystals soaked in or co-crystallized with other molecules allows the determination of where each type of solvent molecule sticks. Molecules that bind tightly to those sites can then be further modified and synthesized and tested for their hepes protease inhibitor activity (J. Travis, Science, 262:1374 (1993)).

In a related approach, iterative drug design is used to identify modifiers of S. aureus FemA activity. Iterative drug design is a method for optimizing associations between a protein and a compound by determining and evaluating the three-dimensional structures of successive sets of protein/compound complexes. In iterative drug design, crystals of a series of protein/compound complexes are obtained and then the three-dimensional structures of each complex is solved. Such an approach provides insight into the association between the proteins and compounds of each complex. This is accomplished by selecting compounds with inhibitory activity, obtaining crystals of this new protein/compound complex, solving the three dimensional structure of the complex, and comparing the associations between the new protein/compound complex and previously solved protein/compound complexes. By observing how changes in the compound affected the protein/compound associations, these associations may be optimized.

Pharmaceutical Compositions

Pharmaceutical compositions of this invention include a potential modifier of S. aureus FemA activity identified according to the invention, or a pharmaceutically acceptable salt thereof, and a pharmaceutically acceptable carrier, adjuvant, or vehicle. The term “pharmaceutically acceptable carrier” refers to a carrier(s) that is “acceptable” in the sense of being compatible with the other ingredients of a composition and not deleterious to the recipient thereof. Optionally, the pH of the formulation is adjusted with pharmaceutically acceptable acids, bases, or buffers to enhance the stability of the formulated compound or its delivery form.

Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. Oral administration or administration by injection is preferred. The term parenteral as used herein includes subcutaneous, intracutaneous, intravenous, intramuscular, intra-articular, intrasynovial, intrasternal, intrathecal, intralesional, and intracranial injection or infusion techniques.

Dosage levels of between about 0.01 and about 100 mg/kg body weight per day, preferably between about 0.5 and about 75 mg/kg body weight per day of the S. aureus FemA inhibitory compounds described herein are useful for the prevention and treatment of S. aureus FemA mediated disease. Typically, the pharmaceutical compositions of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

In order that this invention be more fully understood, the following examples are set forth. These examples are for the purpose of illustration only and are not to be construed as limiting the scope of the invention in any way.

### EXAMPLES

### Example 1

### Analysis of the Structure of S. aureus FemA Expression and Purification

Fermentation of Met-FemA.

To prepare seeds, cells from frozen ampoules were grown in NS86 medium (2.6 g/L K₂HPO₄, 10.9 g/L NaNH₄HPO₄-4H₂O, 2.1 g/L citric acid, 0.67 g/L (NH₄)₂SO₄, 0.25 g/L MgSO₄-7H₂O, 10.4 g/L yeast extract and 5 g/L glycerol) containing ampicillin (100 micrograms/ml) overnight at 37° C. Final densities were 8-12 A₅₅₀. Six 10-liter fermentations were conducted (New Brunswick Microgens) using the following conditions: MIM medium (32 g/L tryptone, 20 g/L yeast extract, 6 g/L Na₂HPO₄, 3 g/L KH2PO4, 0.5 g/L NaCl, and 1 g/L NH₄Cl) containing ampicillin (100 micrograms/ml), 37° C., pH control between 6.9 and 7.3 and dissolved oxygen maintained above 40% saturation. Fermenters were inoculated at a density of 0.1 A₅₅₀; when cells reached a density of approximately 1 A₅₅₀, product expression was induced by the addition of 1 mM IPTG (final concentration). The cells were harvested by ultrafiltration at 2.5 hours post-induction and the retentate centrifuged to isolate the cells.

Fermentation of SeMet-FemA. Cells from frozen ampoules were grown in M9 medium containing ampicillin (100 micrograms/ml) and thiamine (10 micromolar) overnight at 37° C. and reached final densities of approximately 4 A₅₅₀. The fermenter (New Brunswick BioFlo IV), containing 7.5 liters of M9 with ampicillin (100 micrograms/ml) and thiamine (10 micromolar), was inoculated with 400 mL of seed (to an initial density of approximately 0.2 A₅₅₀) and cells grown at 37° C., pH control at 7.0 with dissolved oxygen maintained above 40% saturation. When the cell density reached approximately 0.8 A₅₅₀ methionine biosynthesis was downregulated by the addition of an amino acid cocktail (L-lysine, L-threonine, L-phenylalanine and DL-selenomethionine at final concentrations of 100 micrograms/ml and L-leucine, L-isoleucine and L-valine at final concentrations of 50 micrograms/ml). SeMet-FemA expression was induced by the addition of IPTG (1 mM final concentration) 15 minutes after addition of the amino acids, when the cell density was approximately 1 A₅₅₀. At 3 hours post-induction, the cells were harvested by centrifugation.

Cell Lysis.

A 41.0 gram pellet of methionine-containing FemA and a 70.9 gram pellet of selenomethionine FemA were used in the below described isolations. Frozen cell pellets were thawed at room temperature for approximately 10-15 minutes prior to resuspending in lysis buffer [50 mM Tris-HCl, pH 8.5, 5 mM β-mercaptoethanol, 1.8 g/L lysozyme, 100 mg/L DNAse, 1 per 50 ml protease inhibitor tablets available under the trade designation COMPLETE from Boehringer Manheim] at approximately 6-7 ml of buffer per gram of cell paste. The cell suspensions were kept on ice for 1.5 hours with intermittent mechanical disruption via use of a Tissuemizer. At the end of lysis, the soluble product was separated from insoluble cellular debris by addition of Tergitol 15-S-7 to 4%. The sample was stirred for approximately 30 minutes at 4° C. and then was clarified by centrifugation at approximately 17,000× g at 4° C. for 30 minutes. NaCl was added to 0.5M and imidazole to 25 mM to the clarified lysates.

Immobilized metal affinity chromatography: Clarified cell lysates were loaded onto a column (approximately 1 ml per gram of cell paste; 2.5 cm inside diameter) of Ni⁺²-NTA agarose (Qiagen) which had been pre-washed with water and equilibrated with buffer (50 mM Tris-HCl, 0.5 M NaCl, pH 8.5, 5 mM β-mercaptoethanol); in later isolations, the buffer also included 25 mM imidazole. In each experiment the lysate was loaded onto the column, and then the column was washed with additional buffer until the absorbance returned to baseline. The column was eluted by washing successively with buffer containing 25 mM imidazole, 50 mM imidazole, and then with buffer containing either 250 mM imidazole, or with buffer containing 100 mM imidazole followed by 300 mM imidazole. During the last washes, the eluate was collected in 1-2 minute fractions (approximately 2.5-5.0 ml). Throughout the chromatography the flow rate was 2.5 ml/min, and the absorbance was monitored at 276-278 nm. Fractions were assayed for purity of FemA by SDS-PAGE.

Solubility Studies.

To determine solubility in a variety of buffer systems, 100 microliters of FemA (IMAC purified) was diluted to 1 ml with the buffers of interest. The protein suspensions were placed on an orbital tube rotator at room temperature for 60-75 minutes. Each sample was then passed through a 0.45 micrometer syringe-type filter. The protein content of each filtrate was then estimated as described below.

Anion Exchange Chromatography.

IMAC purified FemA was diluted 1:10 with 50 mM ethanolamine, pH 10.0, 1 mM dithiothreitol) after which it was loaded onto a 20 ml column (2.5 cm inside diameter) of Source 30Q (Amersham-Pharmacia Biotech) anion exchange chromatography medium which had been equilibrated in the same buffer. Following load, each column was washed with additional buffer until the absorbance reached baseline. The columns were then eluted via a linear gradient from 0 to 250 mM NaCl over 10 column volumes. Again, the column was washed and eluted at 2.5 ml/min; the eluate was monitored at 278 nm, and the absorbance was recorded at 2 and 0.2 AUFS. Fractions were assayed for purity of FemA by SDS-PAGE.

Aggregation Studies

Original attempts to work with S. aureus FemA in a pH 8.0 reducing buffer system required the presence of >350 mM NaCl to remain soluble. This preparation would form a haze at 4° C. that could be spun out, but with a loss of protein concentration. Early crystallization attempts were carried out, and were unsuccessful due to the high salt requirement.

By systematic pH screening, it was discovered that Fem A was soluble in low salt concentration at pH 10. A fresh sample, prepared in 50 mM ethanolamine, 1 mM DTT was used for further crystallization attempts. Dynamic light scattering of this sample revealed that it was monodisperse, with a slight amount of multimeric aggregate present. Attempts to freeze and store this sample were unsuccessful, as freezing in any form caused the sample to aggregate heavily in dynamic light scattering measurements. Concentration of this preparation also led to formation of a haze during overnight storage at 4° C. Using preparations of protein that had haze present, simple addition tests of glycerol, NaCl, MgCl₂, and β-octylglucoside were used in an attempt to solubilize the protein. It was discovered that 100 mM NaCl or 20% glycerol would reduce the haze, but that both were required to clarify the solution. No other additives tested had any visible effect.

The inability to completely remove all aggregate (multimeric) species from the Fem A sample resulted in difficulties during screening. Screening attempts usually showed no result (clear) drops or aggregate drops, indicating that the protein was very close to saturation. Crystallization attempts at lower protein concentration were unsuccessful, most likely due to a lack of nucleation centers being present at the lower concentrations. Attempts to increase solubility by addition of glycerol and NaCl as described above resulted in less aggregate observed in the crystallization drops. Crystals were obtained from screening attempts in the presence of glycerol and NaCl, and these crystals were used for structure determination.

Crystallization

Methoinine incorporated S. aureus Fem A was obtained in 50 mM ethanolamine, 1 mM DTT, pH 10.0. The protein was concentrated in an Ultrafree-4 30,000 MWCO filtration unit (Millipore, Bedford, Mass.), to 12 mg/ml. The concentrated sample was used to begin screening Fem A in the crystallization screening library. Dynamic light scattering of the concentrated sample was also used to determine the monodispersity of the preparation. Initially, Hampton Screen I-Lite (Hampton Research, Laguna Niguel, Calif.) was setup at 4° C. and 20° C. using the hanging drop method. No crystals were observed at 20° C., but after 2 weeks at 4° C., crystals were observed growing out of aggregate in condition 36 (4% PEG 8000, 100 mM Tris pH 8.5). The crystals were rod shaped, and were 100-250 micrometers long, and 30-50 micrometers thick. Optimization around these conditions (CS1/F36) was started with the Hampton follow-up library and the 2 crystals repeated, growing to 200 micrometers x 20 micrometers . These crystals were taken to the synchrotron for data collection, where they diffracted to 2.7 Å.

Further work was done in an attempt to solubilize the protein, and it was determined that the addition of 20% glycerol and 100 mM NaCl was necessary for the stability of the protein. Concentration of the protein previous to the addition of glycerol and NaCl usually resulted in a haze that would form in the sample when incubated on ice. Protein was diluted into 20% glycerol and 100 mM NaCl before concentration and then concentrated to 12 mg/mL. Further screening experiments led to discovery of conditions at 20° C. for crystallization. Fem A Tray17 well B2, Hampton Screen 1/condition 6 (30% PEG 4000, 100 mM Tris pH 8.5, 200 mM MgCl₂), grew rod shaped crystals also, but this condition allowed the crystals to grow to over 400 micrometers in the long axis. Precipitation was still present, although in lesser amounts. The precipitation observed might have resulted from the presence of non-specific aggregate, which was observed in low percentages in dynamic light scattering. Another crystal form, which produced thicker rods, was found during a selenomethionine incorporated S. aureus Fem A screen. This condition, Wizard screen I condition 46 from Emerald BioStructures, Bainbridge Island, Wash. (10% PEG 8000, 0.2M Ca(OAc)₂, 0.1M imidazole, pH 8.0), produced crystals suitable for diffraction studies from the screen. The crystal was soaked in a suitable cryoprotectant agent (mother liquor plus 20% glycerol) in 5% glycerol steps and stored in liquid nitrogen until data collection could be performed at the synchrotron.

Structure Determination

One selenomethionine multiple anomalous dispersion (MAD) experiment was performed (2.1 Å resolution) using three different wavelengths (see Table 5). Each of these individual data sets was indexed and integrated separately. The data sets for each experiment were scaled to each other using the program SCALEIT in the CCP4 Program Suite (Collaborative Computational Project N4, Acta Cryst., D50:760-63 (1994)). Patterson maps revealed six selenium sites whose locations were determined by direct methods using SHELX (Sheldrick et al., Acta Cryst., B51:423-31 (1995)). Two pairs of three sites each were tested for authenticity by their ability to generate phases which could identify the other pair of sites in anomalous difference Fourier calculations. A subsequent site was identified by anomalous difference Fourier methods. The seven sites accounted for all of the methionines in the protein including the N-terminal methionine. All heavy atom parameter refinement and phasing calculations were carried out with MLPHARE (Collaborative Computational Project N4, Acta Cryst., D50:760-63 (1994); Otwinowski, “Maximum likelihood refinement of heavy atom parameters” in Isomorphous Replacement and Anomalous Scattering (Wolf, Evans, and Leslie, eds.; Warrington: SERC Daresbury Laboratory) pp. 80-86 (1991)) by treating the remote wavelength as native and the edge and peak wavelengths as derivatives (Ramakrishnan et al., Nature, 362:219-23 (1993)). The phases were subsequently subjected to solvent flattening using the program DM (Cowtan et al., Acta Cryst., D49:148-57 (1993); Cowtan et al., Acta Cryst., D54:487-93 (1998)).

The multiple anomalous dispersion phased electron density map was exceptionally clear. The initial placement of the C_{α} backbone and correlation between the sequence and the main chain was done using the X-AutoFit module in Quanta (Molecular Simulations, San Diego, Calif.). Further model building was done using the program CHAIN (Sack, J. Mol. Graph., 6:224-25 (1988)) and LORE (Finzel, Meth. Enzymol., 277:230-42 (1997)). Because of the high quality of the phases, water molecules were added based on the MAD phased electron density map. Before refinement, the starting R-factor/Free R-factor was 39.5%/40.5%. One cycle of positional refinement, torsion angle dynamics refinement, and individual B factor refinement with a bulk solvent correction led to significant improvement in the model (R-factor/Free R-factor=24.0%/28.2%). The rapid drop in the R-factor during the first cycle of refinement reflected the high quality phases that were determined and used to calculate the initial electron density map. Three more cycles of refinement and rebuilding led to the present model (R-factor/Free R-factor=20.5%/24.5%) (Table 6). All refinement cycles were carried out with XPLOR98 (Brunger, Meth. Mol. Biol., 56:245-66 (1996); Brunger, X-PLOR version 3.1: A system for X-ray Crystallography and NMR. New Haven: Yale Univ. Press (1992)) incorporating bulk solvent correction during the refinement (Jiang et al., J. Mol. Biol., 243:100-15 (1994)). Stereochemistry of the model was checked using PROCHECK (Laskowski et al., J. Appl. Cryst., 26:283-91 (1993)) revealing only two residues in disallowed regions of the Ramachandran plot.

TABLE 6
Refinement Statistics.

| | R-factor | Free R-factor | No. of reflections |
|---|---|---|---|
| 20-2.3 Å F ≧ 2σ | 0.209 | 0.245 | 29,353 |
| | Bonds (Å) | Angles (°) | |
| r.m.s deviation from ideal geometry | 0.009 | 1.34 | |
| | Number of atoms | Average B-factor | |
| Protein | 3305 | 24.5 | |
| Waters | 227 | 33.7 | |
| Total | 3532 | 25.2 | |

FIG. 3 was made using SETOR (Evans, J. Mol. Graphics, 11: 134-38 (1993)), and FIG. 4 was produced with both MOLSCRIPT (Kraulis, J. Appl. Cryst., 24:946-50 (1991)) and Raster 3D (Brotz et al., Eur. J. Biochem., 246:193-99 (1997)), while FIGS. 7, 8, and 9 were produced in MOLSCRIPT (Kraulis, J. Appl. Cryst., 24:946-50 (1991)) alone.

The complete disclosure of all patents, patent applications including provisional applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described; many variations will be apparent to one skilled in the art and are intended to be included within the invention defined by the claims.

### Sequence Listing Free Text

SEQ ID NO:1 staphylococcus aureus FemA protein

SEQ ID NO:2 staphylococcus aureus FemB protein

SEQ ID NO:3 staphylococcus aureus FmhB protein

What is claimed is:

1. A molecule or molecular complex comprising at least a portion of an S. aureus FemA or S. aureus FemA-like substrate binding surface, wherein the substrate binding surface comprises amino acids listed in Table 2, the substrate binding surface being defined by a set of points having a root mean square deviation of less than about 1.5 Å from points representing the backbone atoms of said amino acids as represented by the structure coordinates listed in Table 1.

2. A molecule or molecular complex comprising at least a portion of an S. aureus FemA or S. aureus FemA-like binding site, wherein the binding site comprises the amino acids listed in Table 3, the binding site being defined by a set of points having a root mean square deviation of less than about 1.5 Å from points representing the backbone atoms of said amino acids as represented by the structure coordinates listed in Table 1.

3. The molecule or molecular complex of claim 2, wherein the binding site is a binding site for coenzyme A.

4. A molecule or molecular complex comprising at least a portion of an S. aureus FemA or S. aureus FemA-like binding site, wherein the binding site comprises the amino acids listed in Table 4, the binding site being defined by a set of points having a root mean square deviation of less than about 1.5 Å from points representing the backbone atoms of said amino acids as represented by the structure coordinates listed in Table 1.

5. The molecule or molecular complex of claim 4, wherein the binding site is a binding site for coenzyme A.

6. A molecule or molecular complex that is structurally homologous to an S. aureus FemA molecule or molecular complex, wherein the S. aureus FemA molecule or molecular complex is represented by at least a portion of the structure coordinates listed in Table 1.

7. A scalable three dimensional configuration of points, at least a portion of said points being derived from structure coordinates listed in Table 1 of at least a portion of an S. aureus FemA molecule or molecular complex comprising at least one of a FemA or FemA-like binding site or substrate binding surface.

8. The scalable three dimensional configuration of points of claim 7, wherein substantially all of said points are derived from structure coordinates listed in Table 1 of an S. aureus FemA molecule or molecular complex.

9. The scalable three dimensional configuration of points of claim 7 wherein at least a portion of the points derived from the S. aureus FemA structure coordinates are derived from structure coordinates representing the locations of at least the backbone atoms of amino acids defining an S. aureus FemA substrate binding surface, the substrate binding surface comprising the amino acids listed in Table 2.

10. The scalable three dimensional configuration of points of claim 7 wherein at least a portion of the points derived from the S. aureus FemA structure coordinates are derived from structure coordinates representing the locations of at least the backbone atoms of amino acids defining an S. aureus FemA binding site, the binding site comprising the amino acids listed in Table 3.

11. The scalable three dimensional configuration of points of claim 10, wherein the binding site is a binding site for coenzyme A.

12. The scalable three dimensional configuration of points of claim 7 wherein at least a portion of the points derived from the S. aureus FemA structure coordinates are derived from structure coordinates representing the locations of at least the backbone atoms of amino acids defining an S. aureus FemA binding site, the binding site comprising the amino acids listed in Table 4.

13. The scalable three dimensional configuration of points of claim 12, wherein the binding site is a binding site for coenzyme A.

14. The scalable three dimensional configuration of points of claim 7 displayed as a holographic image, a stereodiagram, a model or a computer-displayed image.

15. A scalable three dimensional configuration of points, at least a portion of the points derived from structure coordinates of at least a portion of a molecule or a molecular complex that is structurally homologous to an S. aureus FemA molecule or molecular complex and comprises at least one of an S. aureus FemA or S. aureus FemA-like binding site or substrate binding surface.

16. The scalable three-dimensional configuration of points of claim 15 displayed as a holographic image, a stereodiagram, a model or a computer-displayed image.

17. A machine-readable data storage medium comprising a data storage material encoded with machine readable data which, when using a machine programmed with instructions for using said data, displays a graphical three-dimensional representation of at least one molecule or molecular complex selected from the group consisting of:
(i) a molecule or molecular complex comprising at least a portion of an S. aureus FemA or S. aureus FemA-like substrate binding surface comprising the amino acids listed in Table 2, the substrate binding surface being defined by a set of points having a root mean square deviation of less than about 1.5 Å from points representing the backbone atoms of said amino acids as represented by structure coordinates listed in Table 1;
(ii) a molecule or molecular complex comprising at least a portion of an S. aureus FemA or S. aureus FemA-like binding site comprising the amino acids listed in Table 3, the binding site being defined by a set of points having a root mean square deviation of less than about 1.5 Å from points representing the backbone atoms of said amino acids as represented by structure coordinates listed in Table 1;
(iii) a molecule or molecular complex comprising at least a portion of an S. aureus FemA or S. aureus FemA-like binding site comprising the amino acids listed in Table 4, the binding site being defined by a set of points having a root mean square deviation of less than about 1.5 Å from points representing the backbone atoms of said amino acids as represented by structure coordinates listed in Table 1; and
(iv) a molecule or molecular complex that is structurally homologous to an S. aureus FemA molecule or molecular complex, wherein the S. aureus FemA molecule or molecular complex is represented by at least a portion of the structure coordinates listed in Table 1.

18. A machine-readable data storage medium comprising a data storage material encoded with a first set of machine readable data which, when combined with a second set of machine readable data, using a machine programmed with instructions for using said first set of data and said second set of data, determines at least a portion of the structure coordinates corresponding to the second set of machine readable data, wherein said first set of data comprises a Fourier transform of at least a portion of the structure coordinates for S. aureus FemA listed in Table 1; and said second set of data comprises an x-ray diffraction pattern of a molecule or molecular complex of unknown structure.

19. A method for obtaining structural information about a molecule or a molecular complex of unknown structure comprising:
crystallizing the molecule or molecular complex;
generating an x-ray diffraction pattern from the crystallized molecule or molecular complex; and
applying at least a portion of the structure coordinates set forth in Table 1 to the x-ray diffraction pattern to generate a three-dimensional electron density map of at least a portion of the molecule or molecular complex whose structure is unknown.

20. A method for homology modeling an S. aureus FemA homolog comprising:
aligning the amino acid sequence of an S. aureus FemA homolog with an amino acid sequence of S. aureus FemA (SEQ ID NO: 1) and incorporating the sequence of the S. aureus FemA homolog into a model of S. aureus FemA formed from structure coordinates set forth in Table 1 to yield a preliminary model of the S. aureus FemA homolog;
subjecting the preliminary model to energy minimization to yield an energy minimized model; and
remodeling regions of the energy minimized model where stereochemistry restraints are violated to yield a final model of the S. aureus FemA homolog.

21. A computer-assisted method for identifying a potential modifier of S. aureus FemA activity comprising:
supplying a computer modeling application with a set of structure coordinates of a molecule or molecular complex, the molecule or molecular complex comprising at least a portion of an S. aureus FemA or S. aureus FemA-like substrate binding surface, the substrate binding surface comprising the amino acids listed in Table 2;
supplying the computer modeling application with a set of structure coordinates of a chemical entity; and
determining whether the chemical entity is expected to bind to or interfere with the molecule or molecular complex, wherein binding to or interfering with the molecule or molecular complex is indicative of potential modification of S. aureus FemA activity.

22. The method of claim 21, wherein the substrate binding surface comprises the amino acids listed in Table 2, the substrate binding surface being defined by a set of points having a root mean square deviation of less than about 1.5 Å from points representing the backbone atoms of said amino acids as represented by structure coordinates listed in Table 1.

23. A computer-assisted method for identifying a potential modifier of S. aureus FemA activity comprising:
supplying a computer modeling application with a set of structure coordinates of a molecule or molecular complex, the molecule or molecular complex comprising at least a portion of an S. aureus FemA or S. aureus FemA-like binding site, the binding site comprising the amino acids listed in Table 3;
supplying the computer modeling application with a set of structure coordinates of a chemical entity; and
determining whether the chemical entity is expected to bind to or interfere with the molecule or molecular complex, wherein binding to or interfering with the molecule or molecular complex is indicative of potential modification of S. aureus FemA activity.

24. The method of claim 23, wherein the binding site comprises the amino acids listed in Table 3, the binding site being defined by a set of points having a root mean square deviation of less than about 1.5 Å from points representing the backbone atoms of said amino acids as represented by structure coordinates listed in Table 1.

25. A computer-assisted method for identifying a potential modifier of S. aureus FemA activity comprising:
supplying a computer modeling application with a set of structure coordinates of a molecule or molecular complex, the molecule or molecular complex comprising at least a portion of an S. aureus FemA or S. aureus FemA-like binding site, the binding site comprising the amino acids listed in Table 4;
supplying the computer modeling application with a set of structure coordinates of a chemical entity; and
determining whether the chemical entity is expected to bind to or interfere with the molecule or molecular complex, wherein binding to or interfering with the molecule or molecular complex is indicative of potential modification of S. aureus FemA activity.

26. The method of claim 25, wherein the binding site comprises the amino acids listed in Table 4, the binding site being defined by a set of points having a root mean square deviation of less than about 1.5 Å from points representing the backbone atoms of said amino acids as represented by structure coordinates listed in Table 1.

27. The method of claim 21, 23, or 25, wherein determining whether the chemical entity is expected to bind to or interfere with the molecule or molecular complex comprises performing a fitting operation between the chemical entity and a binding site or substrate binding surface of the molecule or molecular complex, followed by computationally analyzing the results of the fitting operation to quantify the association between, or the interference with, the chemical entity and the binding site.

28. The method of claim 21, 23, or 25 further comprising screening a library of chemical entities.

29. A computer-assisted method for designing a potential modifier of S. aureus FemA activity comprising:
supplying a computer modeling application with a set of structure coordinates of a molecule or molecular complex, the molecule or molecular complex comprising at least a portion of an S. aureus FemA or S. aureus FemA-like substrate binding surface, the substrate binding surface comprising the amino acids listed in Table 2;
supplying the computer modeling application with a set of structure coordinates for a chemical entity;
evaluating the potential binding or interfering interactions between the chemical entity and the substrate binding surface of the molecule or molecular complex;
structurally modifying the chemical entity to yield a set of structure coordinates for a modified chemical entity; and
determining whether the modified chemical entity is expected to bind to or interfere with the molecule or molecular complex, wherein binding to or interfering with the molecule or molecular complex is indicative of potential modification of S. aureus FemA activity.

30. The method of claim 29, wherein the substrate binding surface comprises the amino acids listed in Table 2, the substrate binding surface being defined by a set of points having a root mean square deviation of less than about 1.5 Å from points representing the backbone atoms of said amino acids as represented by structure coordinates listed in Table 1.

31. A computer-assisted method for designing a potential modifier of S. aureus FemA activity comprising:
supplying a computer modeling application with a set of structure coordinates of a molecule or molecular complex, the molecule or molecular complex comprising at least a portion of an S. aureus FemA or S. aureus FemA-like binding site, the binding site comprising the amino acids listed in Table 3;
supplying the computer modeling application with a set of structure coordinates for a chemical entity;
evaluating the potential binding or interfering interactions between the chemical entity and the binding site of the molecule or molecular complex;
structurally modifying the chemical entity to yield a set of structure coordinates for a modified chemical entity; and
determining whether the modified chemical entity is expected to bind to or interfere with the molecule or molecular complex, wherein binding to or interfering with the molecule or molecular complex is indicative of potential modification of S. aureus FemA activity.

32. The method of claim 31, wherein the binding site comprises the amino acids listed in Table 3, the binding site being defined by a set of points having a root mean square deviation of less than about 1.5 Å from points representing the backbone atoms of said amino acids as represented by structure coordinates listed in Table 1.

33. A computer-assisted method for designing a potential modifier of S. aureus FemA activity comprising:
supplying a computer modeling application with a set of structure coordinates of a molecule or molecular complex, the molecule or molecular complex comprising at least a portion of an S. aureus FemA or S. aureus FemA-like binding site, the binding site comprising the amino acids listed in Table 4;
supplying the computer modeling application with a set of structure coordinates for a chemical entity;
evaluating the potential binding or interfering interactions between the chemical entity and the binding site of the molecule or molecular complex;
structurally modifying the chemical entity to yield a set of structure coordinates for a modified chemical entity; and
determining whether the modified chemical entity is expected to bind to or interfere with the molecule or molecular complex, wherein binding to or interfering with the molecule or molecular complex is indicative of potential modification of S. aureus FemA activity.

34. The method of claim 33, wherein the binding site comprises the amino acids listed in Table 4, the binding site being defined by a set of points having a root mean square deviation of less than about 1.5 Å from points representing the backbone atoms of said amino acids as represented by structure coordinates listed in Table 1.

35. The method of claim 29, 31, or 33, wherein determining whether the modified chemical entity is expected to bind to or interfere with the molecule or molecular complex comprises performing a fitting operation between the chemical entity and a binding site or substrate binding surface of the molecule or molecular complex, followed by computationally analyzing the results of the fitting operation to quantify the association between or interference with the chemical entity and the binding site or substrate binding surface.

36. The method of claim 29, 31, or 33, wherein the set of structure coordinates for the chemical entity is obtained from a chemical fragment library

37. A computer-assisted method for designing a potential modifier of S. aureus FemA activity de novo comprising:
supplying a computer modeling application with a set of structure coordinates of a molecule or molecular complex, the molecule or molecular complex comprising at least a portion of an S. aureus FemA substrate binding surface, wherein the substrate binding surface comprises the amino acids listed in Table 2;
computationally building a chemical entity represented by set of structure coordinates; and
determining whether the chemical entity is expected to bind to or interfere with the molecule or molecular complex, wherein binding to or interfering with the molecule or molecular complex is indicative of potential modification of S. aureus FemA activity.

38. The method of claim 37, wherein the substrate binding surface comprises the amino acids listed in Table 2, the substrate binding surface being defined by a set of points having a root mean square deviation of less than about 1.5 Å from points representing the backbone atoms of said amino acids as represented by structure coordinates listed in Table 1.

39. A computer-assisted method for designing a potential modifier of S. aureus FemA activity de novo comprising:
supplying a computer modeling application with a set of structure coordinates of a molecule or molecular complex, the molecule or molecular complex comprising at least a portion of an S. aureus FemA or S. aureus FemA-like binding site, wherein the binding site comprises the amino acids listed in Table 3;
computationally building a chemical entity represented by set of structure coordinates; and
determining whether the chemical entity is expected to bind to or interfere with the molecule or molecular complex, wherein binding to or interfering with the molecule or molecular complex is indicative of potential modification of S. aureus FemA activity.

40. The method of claim 39, wherein the binding site comprises the amino acids listed in Table 3, the binding site being defined by a set of points having a root mean square deviation of less than about 1.5 Å from points representing the backbone atoms of said amino acids as represented by structure coordinates listed in Table 1.

41. A computer-assisted method for designing a potential modifier of S. aureus FemA activity de novo comprising:
supplying a computer modeling application with a set of structure coordinates of a molecule or molecular complex, the molecule or molecular complex comprising at least a portion of an S. aureus FemA or S. aureus FemA-like binding site, wherein the binding site comprises the amino acids listed in Table 4;
computationally building a chemical entity represented by set of structure coordinates; and
determining whether the chemical entity is expected to bind to or interfere with the molecule or molecular complex, wherein binding to or interfering with the molecule or molecular complex is indicative of potential modification of S. aureus FemA activity.

42. The method of claim 41, wherein the binding site comprises the amino acids listed in Table 4, the binding site being defined by a set of points having a root mean square deviation of less than about 1.5 Å from points representing the backbone atoms of said amino acids as represented by structure coordinates listed in Table 1.

43. The method of claim 37, 39, or 41, wherein determining whether the chemical entity is expected to bind to or interfere with the molecule or molecular complex comprises performing a fitting operation between the chemical entity and a binding site of the molecule or molecular complex, followed by computationally analyzing the results of the fitting operation to quantify the association between or interference with the chemical entity and the binding site.

44. The method of any of claims 21, 23, 25, 29, 31, 33, 37, 39, or 41 further comprising supplying or synthesizing the chemical entity, then assaying the chemical entity to determine whether it modifies S. aureus FemA activity.

45. A method for making a potential modifier of S. aureus FemA activity, the method comprising chemically or enzymatically synthesizing a chemical entity to yield a potential modifier of S. aureus FemA activity, the chemical entity having been identified during a computer-assisted process comprising supplying a computer modeling application with a set of structure coordinates of a molecule or molecular complex, the molecule or molecular complex comprising at least a portion of a S. aureus FemA or S. aureus FemA-like binding site or substrate binding surface; supplying the computer modeling application with a set of structure coordinates of a chemical entity; and determining whether the chemical entity is expected to bind to or interfere with the molecule or molecular complex at a binding site or substrate binding surface, wherein binding to or interfering with the molecule or molecular complex is indicative of potential modification of S. aureus FemA activity.

46. A method for making a potential modifier of S. aureus FemA activity, the method comprising chemically or enzymatically synthesizing a chemical entity to yield a potential modifier of S. aureus FemA activity, the chemical entity having been designed during a computer-assisted process comprising supplying a computer modeling application with a set of structure coordinates of a molecule or molecular complex, the molecule or molecular complex comprising at least a portion of a S. aureus FemA or S. aureus FemA-like binding site or substrate binding surface; supplying the computer modeling application with a set of structure coordinates for a chemical entity; evaluating the potential binding interactions between the chemical entity and a binding site or substrate binding surface of the molecule or molecular complex; structurally modifying the chemical entity to yield a set of structure coordinates for a modified chemical entity; and determining whether the chemical entity is expected to bind to or interfere with the molecule or molecular complex at the binding site, wherein binding to or interfering with the molecule or molecular complex is indicative of potential modification of S. aureus FemA activity.

47. A method for making a potential modifier of S. aureus FemA activity, the method comprising chemically or enzymatically synthesizing a chemical entity to yield a potential modifier of S. aureus FemA activity, the chemical entity having been designed during a computer-assisted process comprising supplying a computer modeling application with a set of structure coordinates of a molecule or molecular complex, the molecule or molecular complex comprising at least a portion of a S. aureus FemA or S. aureus FemA-like binding site or substrate binding surface; computationally building a chemical entity represented by set of structure coordinates; and determining whether the chemical entity is expected to bind to or interfere with the molecule or molecular complex at a binding site or substrate binding surface, wherein binding to or interfering with the molecule or molecular complex is indicative of potential modification of S. aureus FemA activity.

48. A potential modifier of S. aureus FemA activity identified, designed or made according to the method of any of the claims 21, 23, 25, 29, 31, 33, 37, 39, 41, 45, 46, or 47.

49. A composition comprising a potential modifier of S. aureus FemA activity identified, designed or made according to the method of any of claims 21, 23, 25, 29,31, 33, 37, 39, 41, 45, 46, or 47.

50. A pharmaceutical composition comprising a potential modifier of S. aureus FemA activity identified or designed according to the method of any of claims 21, 23, 25, 29, 31, 33, 37, 39, 41, 45, 46, or 47 or a salt thereof, and pharmaceutically acceptable carrier.

51. A method for crystallizing an S. aureus FemA molecule or molecular complex comprising:
preparing purified S. aureus FemA at a concentration of about 1 mg/ml to about 50 mg/ml; and
crystallizing S. aureus FemA from a solution comprising about 1 wt. % to about 50 wt. % PEG, 0 wt. % to about 50 wt. % glycerol, 0 M to about 1 M NaCl, 0 wt. % to about 40 wt. % DMSO, about 100 mM to about 1 M Ca(OAc)₂ and/or MgCl₂, and buffered to a pH of about 7 to about 10.

52. A crystal of S. aureus FemA.

53. The crystal of claim 52 having the orthorhombic space group symmetry P2₁2₁2₁.

54. The crystal of claim 52 comprising a unit cell having dimensions a, b, and c; wherein a is about 40 Å to about 70 Å, b is about 75 Å to about 105 Å, and c is about 95 Å to about 125 Å; and α=β=γ=90°.

55. The crystal of claim 52 comprising atoms arranged in a spatial relationship represented by the structure coordinates listed in Table 1.

56. The crystal of claim 52 including amino acids having the sequence SEQ ID NO: 1.

57. The crystal of claim 52 having amino acid sequence SEQ ID NO: 1, with the proviso that at least one methionine is replaced with selenomethionine.
